(19) United States
(12) Patent Application Publication
(10) Pub. No.: US 2022/0194936 A1
(43) Pub. Date: Jun. 23, 2022

(54) CRYSTALLINE FORMS OF N-(5-(5-((1R,2S)-2-FLUOROCYCLOPROPYL)-1,2,4-OXADIAZOL-3-YL)-2-METHYLPHENYL)IMIDAZO[1,2-A]PYRIDINE-3-CARBOXAMIDE

(71) Applicant: Novartis AG, Basel (CH)

(72) Inventors: Xiaoyang WANG, Shanghai (CN); Andreas KORDIKOWSKI, Basel (CH)

(21) Appl. No.: 17/595,260

(22) PCT Filed: May 13, 2020
(86) PCT No.: PCT/CN2020/090060

§ 371 Date: Nov. 12, 2021

(30) Foreign Application Priority Data
May 13, 2019 (CN) PCT/CN2019/086582

(51) Int. Cl. C07D 471/04; C07C 57/15

(52) U.S. Cl. CPC C07D 471/04; C07B 2200/13; C07C 57/15

(57) ABSTRACT

Provided are crystalline forms of N-(5{5-((1R,2S)-2-fluorocyclopropyl)-1,2,4-oxadiazol-3-yl)-2-methylphenyl)imidazo[1,2-a]pyridine-3-carboxamide fumaric acid, in particular Form A and a N-(5{5-((1R,2S)-2-fluorocyclo-propyl)-1,2,4-oxadiazol-3-yl)-2-methylphenyl)imidazo[1,2-a]pyridine-3-carboxamide fumaric acid co-crystal. Provided are also the processes for preparation of such crystalline forms. Furthermore, Provided is a pharmaceutical composition comprising said N-(5{5-((1R,2S)-2-fluorocyclopropyl)-1,2,4-oxadiazol-3-yl)-2-methylphenyl)imidazo[1,2-a]pyridine-3-carboxamide fumaric acid Form A, or said N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, and at least one pharmaceutically acceptable excipient. The pharmaceutical composition can be used as a medicament, in particular for the treatment and/or prophylaxis of a mast-cell associated disease, a respiratory disease, an inflammatory disorder, irritable bowel syndrome (IBS), inflammatory bowel disease (IBD), an autoimmune disorder, a metabolic disease, a fibrosis disease.

### FIELD OF THE INVENTION

The present invention relates to crystalline forms of N-(5-(5-((1R,2S)-2-fluorocyclopropyl)-1,2,4-oxadiazol-3-yl)-2-methylphenyl)imidazo[1,2-a]pyridine-3-carboxamide. The present invention further relates to the process for the preparation of the crystalline forms of N-(5-(5-((1R,2S)-2-fluorocyclopropyl)-1,2,4-oxadiazol-3-yl)-2-methylphenyl)imidazo[1,2-a]pyridine-3-carboxamide. Furthermore, the invention relates to a pharmaceutical composition comprising said crystalline forms of N-(5-(5-((1R,2S)-2-fluorocyclopropyl)-1,2,4-oxadiazol-3-yl)-2-methylphenyl)imidazo[1,2-a]pyridine-3-carboxamide and at least one pharmaceutically acceptable excipient. The pharmaceutical composition of the present invention can be used as a medicament, in particular for the treatment a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) and primary pulmonary hypertension (PPH). In particular, the pharmaceutical compositions of the present invention can be used as a medicament for the treatment and/or prophylaxis of asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type 11 diabetes.

### BACKGROUND OF THE INVENTION

N-(5-(5-((1R,2S)-2-fluorocyclopropyl)-1,2,4-oxadiazol-3-yl)-2-methylphenyl)imidazo[1,2-a]pyridine-3-carboxamide is a selective inhibitor of c-kit kinase, useful for the depletion of mast cells and thus is useful for treating a mast-cell associated disease including asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type II diabetes.

N-(5-(5-((1R,2S)-2-fluorocyclopropyl)-1,2,4-oxadiazol-3-yl)-2-methylphenyl)imidazo[1,2-a]pyridine-3-carboxamide can be represented by the chemical structure as depicted in Formula (A):

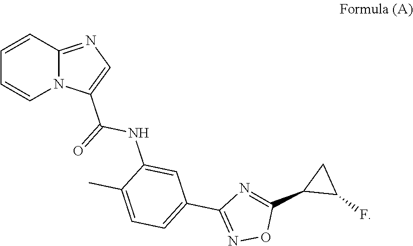 [SMILES: Cc1ccc(-c2noc([C@H]3C[C@@H]3F)n2)cc1NC(=O)c1cnc2ccccn12]

N-(5-(5-((1R,2S)-2-fluorocyclopropyl)-1,2,4-oxadiazol-3-yl)-2-methylphenyl)imidazo[1,2-a]pyridine-3-carboxamide is disclosed in WO 2013/033070 A1 as the free form compound.

WO 2013/033070 A1 generically discloses several acid addition salts of N-(5-(5-((1R,2S)-2-fluorocyclopropyl)-1,2,4-oxadiazol-3-yl)-2-methylphenyl)imidazo[1,2-a]pyridine-3-carboxamide, such as salts with hydrobromic acid, hydrochloric acid, sulfuric acid, nitric acid, phosphoric acid, succinic acid, maleic acid, malonic acid, mandelic acid, acetic acid, propionic acid, glycolic acid, oxalic acid, fumaric acid, citric acid, tartaric acid, lactic acid, benzoic acid, salicylic acid, glutamic acid, aspartic acid, toluenesulfonic acid, sulfosalicylic acid, benzenesulfonic acid, methanesulfonic acid, ethanesulfonic acid, naphthalenesulfonic acid, such as 2-naphthalenesulfonic acid, or hexanoic acid, although the fumaric acid salt is not specifically disclosed.

Co-crystals are structurally readily distinguishable from salts because unlike salts, their components are in a neutral state and interact nonionically. In addition, co-crystals structurally differ from polymorphs, which are defined as including only single-component crystalline forms that have different arrangements or conformations of the molecules in the crystal lattice Instead, co-crystals are structurally more similar to solvates and hydrates, in that both contain more than one component in the crystal lattice and the interaction between these components is nonionic. From a physical chemistry perspective, co-crystals can be viewed as a special case of solvates and hydrates, wherein the second component, the co-crystal former, is nonvolatile. (see also “Regulatory Classification of Pharmaceutical Co-Crystals”, Guidance for Industry, FDA, Revision 1, August 2016).

Different solid-state forms of an active pharmaceutical ingredient (API) often possess different physical and chemical properties such as but not limited to dissolution rate, solubility, chemical stability, physical stability, hygroscopicity, melting point, morphology, flowability, bulk density and compressibility. Apart from conventional solid-state forms of an API, such as polymorphs, pseudopolymorphs (hydrates and solvates) and salts, pharmaceutical co-crystals open up further opportunities for customizing the physicochemical properties of APIs with a process or clinical need. For example, they can be tailored to enhance drug product bioavailability and stability and to enhance the processability of APIs during drug product manufacture.

The tendency of a drug substance to absorb water from the environment can negatively affect the pharmaceutical behavior and quality of a drug product. Water absorption for example can lead to chemical degradation (e.g. via hydrolysis), trigger changes of the physical form (e.g. via hydrate formation), lead to changes in dissolution behavior and influence powder properties such as flowability, compactability, tableting and compression behavior etc.

Furthermore, the sudden appearance or disappearance of a metastable polymorph can present a problem in process development. Similarly, serious pharmaceutical consequences arise if solid state transformations occur in a dosage form.

There is thus a need to provide a solid state form of N-(5-(5-((1R,2S)-2-fluorocyclopropyl)-1,2,4-oxadiazol-3-yl)-2-methylphenyl)imidazo[1,2-a]pyridine-3-carboxamide, which possess physicochemical properties allowing for the reliable production of a safe and efficacious drug product comprising N-(5-(5-((1R,2S)-2-fluorocyclopropyl)-1,2,4-oxadiazol-3-yl)-2-methylphenyl)imidazo[1,2-a]pyridine-3-carboxamide. It is also an objective of the present invention to provide a fumaric acid salt or co-crystal of N-(5-(5-((1R,2S)-2-fluorocyclopropyl)-1,2,4-oxadiazol-3-yl)-2-methylphenyl)imidazo[1,2-a]pyridine-3-carboxamide.

### SUMMARY OF THE INVENTION

The present invention solves one or more of the above mentioned problems by providing a crystalline form of N-(5-(5-((1R,2S)-2-fluorocyclopropyl)-1,2,4-oxadiazol-3-yl)-2-methylphenyl)imidazo[1,2-a]pyridine-3-carboxamide, which is hereinafter also referred to “Form A”. “Form A” of N-(5-(5-((1R,2S)-2-fluorocyclopropyl)-1,2,4-oxadiazol-3-yl)-2-methylphenyl)imidazo[1,2-a]pyridine-3-carboxamide of the present invention possesses favorable physicochemical properties for a drug substance intended for use in an oral solid dosage form. Said properties include chemical stability, physical stability, hygroscopicity, solubility, dissolution, morphology, crystallinity, flowability, compactability and wettability.

In particular, the crystalline Form A of N-(5-(5-((1R,2S)-2-fluorocyclopropyl)-1,2,4-oxadiazol-3-yl)-2-methylphenyl)imidazo[1,2-a]pyridine-3-carboxamide of the present invention preserves its crystal structure even when subjected to severe temperature and/or humidity stress conditions or when slurried for prolonged time in various solvents. The usage of the thermodynamically stable form of a compound is highly appreciated as polymorphic conversions, which may occur during manufacturing process and storage of a drug substance can be excluded, when the stable form is used. This ensures reliable bioavailability and therefore consistent efficacy of a drug product.

The invention also provides a pharmaceutical co-crystal of N-(5-(5-((1R,2S)-2-fluorocyclopropyl)-1,2,4-oxadiazol-3-yl)-2-methylphenyl)imidazo[1,2-a]pyridine-3-carboxamide, in particular, the invention provides a pharmaceutical co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid.

Abbreviations

PXRD powder X-ray diffractogram

DSC differential scanning calorimetry

TGA thermogravimetric analysis

NMR nuclear magnetic resonance

RT room temperature

RH relative humidity

API active pharmaceutical ingredient

### Definitions

The terms “N-(5-(5-((1R,2S)-2-fluorocyclopropyl)-1,2,4-oxadiazol-3-yl)-2-methylphenyl)imidazo[1,2-a]pyridine-3-carboxamide form A” or “Form A of N-(5-(5-((1R,2S)-2-fluorocyclopropyl)-1,2,4-oxadiazol-3-yl)-2-methylphenyl)imidazo[1,2-a]pyridine-3-carboxamide” as used herein, refers to the crystalline form of N-(5-(5-((1R,2S)-2-fluorocyclopropyl)-1,2,4-oxadiazol-3-yl)-2-methylphenyl)imidazo[1,2-a]pyridine-3-carboxamide. Form A of N-(5-(5-((1R,2S)-2-fluorocyclopropyl)-1,2,4-oxadiazol-3-yl)-2-methylphenyl)imidazo[1,2-a]pyridine-3-carboxamide can be characterized by having a powder X-ray diffractogram comprising reflections at 2-Theta angles of (5.0±0.2)° and (13.2±0.2)°, when measured at a temperature in the range of from 20 to 30° C. with Cu-Kalpha radiation having a wavelength of 0.15406 nm.

The terms “N-(5-(5-((1R,2S)-2-fluorocyclopropyl)-1,2,4-oxadiazol-3-yl)-2-methylphenyl)imidazo[1,2-a]pyridine-3-carboxamide form HA” or “Form HA of N-(5-(5-((1R,2S)-2-fluorocyclopropyl)-1,2,4-oxadiazol-3-yl)-2-methylphenyl)imidazo[1,2-a]pyridine-3-carboxamide” as used herein, refers to the crystalline form of N-(5-(5-((1R,2S)-2-fluorocyclopropyl)-1,2,4-oxadiazol-3-yl)-2-methylphenyl)imidazo[1,2-a]pyridine-3-carboxamide. Form A of N-(5-(5-((1R,2S)-2-fluorocyclopropyl)-1,2,4-oxadiazol-3-yl)-2-methylphenyl)imidazo[1,2-a]pyridine-3-carboxamide can be characterized by having a powder X-ray diffractogram comprising reflections at 2-Theta angles of 6.7±0.2) ° and (18.0±0.2)°, when measured at a temperature in the range of from 20 to 30° C. with Cu-Kalpha radiation having a wavelength of 0.15406 nm.

The terms “N-(5-(5-((1R,2S)-2-fluorocyclopropyl)-1,2,4-oxadiazol-3-yl)-2-methylphenyl)imidazo[1,2-a]pyridine-3-carboxamide form HB” or “Form HB of N-(5-(5-((1R,2S)-2-fluorocyclopropyl)-1,2,4-oxadiazol-3-yl)-2-methylphenyl)imidazo[1,2-a]pyridine-3-carboxamide” as used herein, refers to the crystalline form of N-(5-(5-((1R,2S)-2-fluorocyclopropyl)-1,2,4-oxadiazol-3-yl)-2-methylphenyl)imidazo[1,2-a]pyridine-3-carboxamide. Form A of N-(5-(5-((1R,2S)-2-fluorocyclopropyl)-1,2,4-oxadiazol-3-yl)-2-methylphenyl)imidazo[1,2-a]pyridine-3-carboxamide can be characterized by having a powder X-ray diffractogram comprising reflections at 2-Theta angles of (6.7±0.2) ° and (18.0±0.2)°, when measured at a temperature in the range of from 20 to 30° C. with Cu-Kalpha radiation having a wavelength of 0.15406 nm.

The term “co-crystal” as used herein refers to crystalline materials composed of two or more different molecular and/or ionic compounds in the same crystal lattice that are associated by nonionic and noncovalent bonds, wherein at least two of the individual molecular and/or ionic compounds are solids at room temperature.

The terms “N-(5-(5-((1R,2S)-2-fluorocyclopropyl)-1,2,4-oxadiazol-3-yl)-2-methylphenyl)imidazo[1,2-a]pyridine-3-carboxamide co-crystal with fumaric acid” or “co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid” or “N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal” as used interchangeably herein refer to a crystalline compound comprising N-(5-(5-((1R,2S)-2-fluorocyclopropyl)-1,2,4-oxadiazol-3-yl)-2-methylphenyl)imidazo[1,2-a]pyridine-3-carboxamide as active pharmaceutical ingredient and fumaric acid as co-crystal former in the same crystal lattice, wherein the interaction between N-(5-(5-((1R,2S)-2-fluorocyclopropyl)-1,2,4-oxadiazol-3-yl)-2-methylphenyl)imidazo[1,2-a]pyridine-3-carboxamide and fumaric acid is of nonionic and noncovalent nature.

The term “N-(5-(5-((1R,2S)-2-fluorocyclopropyl)-1,2,4-oxadiazol-3-yl)-2-methylphenyl)imidazo[1,2-a]pyridine-3-carboxamide form A” as used herein, refers to the crystalline form of N-(5-(5-((1R,2S)-2-fluorocyclopropyl)-1,2,4-oxadiazol-3-yl)-2-methylphenyl)imidazo[1,2-a]pyridine-3-carboxamide. Form A of N-(5-(5-((1R,2S)-2-fluorocyclopropyl)-1,2,4-oxadiazol-3-yl)-2-methylphenyl)imidazo[1,2-a]pyridine-3-carboxamide can be characterized by having a powder X-ray diffractogram (PXRD) comprising reflections at 2-Theta angles of (13.2±0.2) ° and (19.7±0.2)°, when measured at a temperature in the range of from 20 to 30° C. with Cu-Kalpha radiation having a wavelength of 0.15406 nm.

As used herein the term “room temperature” refers to a temperature in the range of from 20 to 30° C.

As used herein, the term “measured at a temperature in the range of from 20 to 30° C.” refers to a measurement under standard conditions. Typically, standard conditions mean a temperature in the range of from 20 to 30° C., i.e. at room temperature. Standard conditions can mean a temperature of about 22° C. Typically, standard conditions can additionally mean a measurement under 20-50% relative humidity.

The term “reflection” with regard to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order.

Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitaij K. Pecharsky and Peter Y. Zavalij, Kluwer Academic Publishers, 2003, page 3).

The term “essentially the same” with reference to powder X-ray diffraction means that variabilities in reflection positions and relative intensities of the reflections are to be taken into account. For example, a typical precision of the 2-Theta values is in the range of ±0.2° 2-Theta, preferably in the range of ±0.1° 2-Theta. Thus, a reflection that usually appears at 3.6° 2-Theta for example can appear between 3.4° and 3.8° 2-Theta, preferably between 3.5 and 3.7° 2-Theta on most X-ray diffractometers under standard conditions. Furthermore, one skilled in the art will appreciate that relative reflection intensities will show inter-apparatus variability as well as variability due to degree of crystallinity, preferred orientation, sample preparation and other factors known to those skilled in the art and should be taken as qualitative measure only.

The terms “solid form” or “solid-state form” as used herein refers to any crystalline and/or amorphous phase of a compound. Crystalline phases include anhydrous/non-solvated forms of a compound and their polymorphs, hydrates and solvates of a compound and their polymorphs, salts and co-crystals of a compound and any mixtures thereof.

As used herein, the term “amorphous” refers to a solid form of a compound that is not crystalline. An amorphous compound possesses no long-range order and does not display a definitive X-ray diffraction pattern with reflections.

As used herein the term “polymorph” refers to crystalline forms having the same chemical composition but different spatial arrangements of the molecules, atoms, and/or ions forming the crystal.

The terms “anhydrous” or “anhydrate” as used herein refer to a crystalline solid where no water is cooperated in or accommodated by the crystal structure. Anhydrous forms may still contain residual water, which is not part of the crystal structure but may be adsorbed on the surface or absorbed in disordered regions of the crystal. Typically, an anhydrous form does not contain more than 2.0 weight %, preferably not more than 1.0 weight % based on the weight of the crystalline form. The water content can be determined by Karl-Fischer Coulometry and/or by thermogravimetric analysis (TGA), e.g. by determining the mass loss in the range of from 25 to 180° C., 190° C. or 200° C. at a heating rate of 10° C./min.

The term “hydrate” as used herein, refers to a crystalline solid where either water is cooperated in or accommodated by the crystal structure e.g. is part of the crystal structure or entrapped into the crystal (water inclusions). Thereby, water can be present in a stoichiometric or non-stoichiometric amount. When water is present in stoichiometric amount, the hydrate may be referred to by adding greek numeral prefixes. For example, a hydrate may be referred to as a hemihydrate or as a monohydrate depending on the water/compound stoichiometry. The water content can be measured, for example, by Karl-Fischer-Coulometry.

The terms “dehydrating” or “dehydration” as used herein, describe the at least partial removal of water from the crystal structure of the host molecule.

The term “solvate” as used herein, refers to a crystalline solid were either one or more organic solvent(s) is/are cooperated in or accommodated by the crystal structure e.g. is/are part of the crystal structure or entrapped into the crystal (water inclusions). Thereby, the one or more organic solvent(s) can be present in a stoichiometric or non-stoichiometric amount. When the one or more organic solvent(s) is/are present in stoichiometric amount(s), the solvate may be referred to by adding greek numeral prefixes. For example, a solvate may be referred to as a hemisolvate or as a monosolvate depending on the solvent(s)/compound stoichiometry. The solvent content can be measured, for example, by GC, NMR, SXRD and/or TGA/MS.

The term “non-solvated” as used herein, when talking about a crystalline solid indicates that no organic solvent is cooperated in or accommodated by the crystal structure. Non-solvated forms may still contain residual organic solvents, which are not part of the crystal structure but may be adsorbed on the surface or absorbed in disordered regions of the crystal. Typically, a non-solvated form does not contain more than 2.0 weight %, preferably not more than 1.0 weight %, and most preferably not more than 0.5 weight % of organic solvents, based on the weight of the crystalline form. The organic solvent content can be determined by thermogravimetric analysis (TGA), e.g. by determining the mass loss in the range of from 25 to 180° C., 190° C. or 200° C. at a heating rate of 10° C./min or by 1H-NMR.

The term “isostructural solvate” as used herein, refers to solvates having the same space group with only small distortions of the unit cell dimensions and the same type of molecular network of the host molecule. Isostructural solvates as defined herein, differ in the type of organic solvent(s) present as guest molecule(s).

The terms “desolvating” or “desolvation” as used herein, describe the at least partial removal of organic solvent from the crystal structure of the host molecule.

The N-(5-(5-((1R,2S)-2-fluorocyclopropyl)-1,2,4-oxadiazol-3-yl)-2-methylphenyl)imidazo[1,2-a]pyridine-3-carboxamide Form A may be referred to herein as being characterized by a powder X-ray diffractogram “as shown in” a figure. The person skilled in the art understands that factors such as variations in instrument type, response and variations in sample directionality, sample concentration, sample purity, sample history and sample preparation may lead to variations, for example relating to the exact reflection or peak positions and intensities. However, a comparison of the graphical data in the figures herein with the graphical data generated for an unknown physical form and the confirmation that two sets of graphical data relate to the same crystal form is well within the knowledge of a person skilled in the art.

The N-(5-(5-((1R,2S)-2-fluorocyclopropyl)-1,2,4-oxadiazol-3-yl)-2-methylphenyl)imidazo[1,2-a]pyridine-3-carboxamide Form HA may be referred to herein as being characterized by a powder X-ray diffractogram “as shown in” a figure. The person skilled in the art understands that factors such as variations in instrument type, response and variations in sample directionality, sample concentration, sample purity, sample history and sample preparation may lead to variations, for example relating to the exact reflection or peak positions and intensities. However, a comparison of the graphical data in the figures herein with the graphical data generated for an unknown physical form and the confirmation that two sets of graphical data relate to the same crystal form is well within the knowledge of a person skilled in the art.

The N-(5-(5-((1R,2S)-2-fluorocyclopropyl)-1,2,4-oxadiazol-3-yl)-2-methylphenyl)imidazo[1,2-a]pyridine-3-carboxamide Form HB may be referred to herein as being characterized by a powder X-ray diffractogram “as shown in” a figure. The person skilled in the art understands that factors such as variations in instrument type, response and variations in sample directionality, sample concentration, sample purity, sample history and sample preparation may lead to variations, for example relating to the exact reflection or peak positions and intensities. However, a comparison of the graphical data in the figures herein with the graphical data generated for an unknown physical form and the confirmation that two sets of graphical data relate to the same crystal form is well within the knowledge of a person skilled in the art.

The N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal may be referred to herein as being characterized by a powder X-ray diffractogram (PXRD) “as shown in” a figure. The person skilled in the art understands that factors such as variations in instrument type, response and variations in sample directionality, sample concentration, sample purity, sample history and sample preparation may lead to variations, for example relating to the exact reflection or peak positions and intensities. However, a comparison of the graphical data in the figures herein with the graphical data generated for an unknown physical form and the confirmation that two sets of graphical data relate to the same crystal form is well within the knowledge of a person skilled in the art.

As used herein, the term “mother liquor” refers to the solution remaining after crystallization of a solid from said solution.

A “predetermined amount” as used herein with regard to N-(5-(5-((1R,2S)-2-fluorocyclopropyl)-1,2,4-oxadiazol-3-yl)-2-methylphenyl)imidazo[1,2-a]pyridine-3-carboxamide Form A or co-crystal of the present invention refers to the initial amount of the N-(5-(5-((1R,2S)-2-fluorocyclopropyl)-1,2,4-oxadiazol-3-yl)-2-methylphenyl)imidazo[1,2-a]pyridine-3-carboxamide Form A or co-crystal used for the preparation of a pharmaceutical composition having a desired dosage strength of N-(5-(5-((1R,2S)-2-fluorocyclopropyl)-1,2,4-oxadiazol-3-yl)-2-methylphenyl)imidazo[1,2-a]pyridine-3-carboxamide.

As used herein, the term “effective amount” in conjunction with the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention encompasses an amount of the N-(5-(5-((1R,2S)-2-fluorocyclopropyl)-1,2,4-oxadiazol-3-yl)-2-methylphenyl)imidazo[1,2-a]pyridine-3-carboxamide Form A or co-crystal which causes the desired therapeutic or prophylactic effect.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

As used herein, the term “essentially free of any other solid-state form” with reference to the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention, means that the N-(5-(5-((1R,2S)-2-fluorocyclopropyl)-1,2,4-oxadiazol-3-yl)-2-methylphenyl)imidazo[1,2-a]pyridine-3-carboxamide Form A contains at most 20 weight %, preferably at most 10 weight %, more preferably at most 5 weight %, 4 weight %, 3 weight %, 2 weight % or 1 weight % of any other solid-state form of N-(5-(5-((1R,2S)-2-fluorocyclopropyl)-1,2,4-oxadiazol-3-yl)-2-methylphenyl)imidazo[1,2-a]pyridine-3-carboxamide, in particular N-(5-(5-((1R,2S)-2-fluorocyclopropyl)-1,2,4-oxadiazol-3-yl)-2-methylphenyl)imidazo[1,2-a]pyridine-3-carboxamide form HA or Form HB, based on the weight of the composition.

The term “pharmaceutically acceptable excipient” as used herein refers to substances, which do not show a significant pharmacological activity at the given dose and that are added to a pharmaceutical composition in addition to the active pharmaceutical ingredient. Excipients may take the function of vehicle, diluent, release agent, disintegrating agent, dissolution modifying agent, absorption enhancer, stabilizer or a manufacturing aid among others. Excipients may include fillers (diluents), binders, disintegrants, lubricants and glidants.

The terms “filler” or “diluent” as used herein refer to substances that are used to dilute the active pharmaceutical ingredient prior to delivery. Diluents and fillers can also serve as stabilizers.

As used herein the term “binder” refers to substances which bind the active pharmaceutical ingredient and pharmaceutically acceptable excipient together to maintain cohesive and discrete portions.

The terms “disintegrant” or “disintegrating agent” as used herein refers to substances which, upon addition to a solid pharmaceutical composition, facilitate its break-up or disintegration after administration and permits the release of the active pharmaceutical ingredient as efficiently as possible to allow for its rapid dissolution.

The term “lubricant” as used herein refers to substances which are added to a powder blend to prevent the compacted powder mass from sticking to the equipment during tableting or encapsulation process. They aid the ejection of the tablet from the dies and can improve powder flow.

The term “glidant” as used herein refers to substances which are used for tablet and capsule formulations in order to improve flow properties during tablet compression and to produce an anti-caking effect.

The term “photostabilizing agent” as used herein refers to substances which prevent or reduce the photodegradation or photodecomposition of the active pharmaceutical ingredient upon light exposure. In other words, the photostabilizing agent functions to prevent or reduce the formation of photodegradation products. Typically, the photostabilizing agent prevents or reduces the photodegradation of the light sensitive active pharmaceutical ingredient by blocking or reducing the exposure of the molecule to light within a wavelength range.

As used herein, the term “effective amount” in conjunction with a photostabilizing agent encompasses an amount of the photostabilizing agent which is sufficient to prevent or reduce the photodegradation of the active pharmaceutical ingredient, such that the amount of photodegradation products that is produced is limited to a desired maximum level under specific light conditions.

### BRIEF DESCRIPTION OF THE FIGURES

FIG. 1: illustrates a representative powder X-ray diffractogram (PXRD) curve of Form A of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the scattering angle in °2-Theta, the y-axis shows the intensity of the scattered X-ray beam in counts of detected photons.

FIG. 2: illustrates a representative differential scanning calorimetry (DSC) curve of Form A of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the temperature in degree Celsius (° C.), the y-axis shows the heat flow rate in Watt per gram (W/g) with endothermic peaks going up.

FIG. 3: illustrates a representative thermogravimetric analysis (TGA) curve of Form A of N-(5-(5-((1R,2S)-2-fluorocyclopropyl)-1,2,4-oxadiazol-3-yl)-2-methylphenyl)imidazo[1.2-a]pyridine-3-carboxamide according to the present invention. The x-axis shows the temperature in degree Celsius (° C.), the y-axis shows the mass (loss) of the sample in weight percent (weight %).

FIG. 4: illustrates a representative powder X-ray diffractogram (PXRD) curve Form HA of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the scattering angle in °2-Theta, the y-axis shows the intensity of the scattered X-ray beam in counts of detected photons.

FIG. 5: illustrates a representative differential scanning calorimetry (DSC) curve of Form HA N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the temperature in degree Celsius (° C.), the y-axis shows the heat flow rate in Watt per gram (W/g) with endothermic peaks going up.

FIG. 6: illustrates a representative thermogravimetric analysis (TGA) curve of Form HA of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the temperature in degree Celsius (° C.), the y-axis shows the mass (loss) of the sample in weight percent (weight %).

FIG. 7: illustrates a representative powder X-ray diffractogram (PXRD) curve Form HB of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the scattering angle in °2-Theta, the y-axis shows the intensity of the scattered X-ray beam in counts of detected photons.

FIG. 8: illustrates a representative differential scanning calorimetry (DSC) curve of Form HB of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the temperature in degree Celsius (° C.), the y-axis shows the heat flow rate in Watt per gram (W/g) with endothermic peaks going up.

FIG. 9: illustrates a representative thermogravimetric analysis (TGA) curve of Form HB of N-(5-(5-((1R,2S)-2-fluorocyclopropyl)-1,2,4-oxadiazol-3-yl)-2-methylphenyl)imidazo[1,2-a]pyridine-3-carboxamide according to the present invention. The x-axis shows the temperature in degree Celsius (° C.), the y-axis shows the mass (loss) of the sample in weight percent (weight %).

FIG. 10: illustrates a representative powder X-ray diffractogram (PXRD) of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention. The x-axis shows the scattering angle in °2-Theta, the y-axis shows the intensity of the scattered X-ray beam in counts of detected photons.

FIG. 11: illustrates a representative differential scanning calorimetry (DSC) curve of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention. The x-axis shows the temperature in degree Celsius (° C.), the y-axis shows the heat flow rate in Watt per gram (W/g).

FIG. 12: illustrates a representative thermogravimetric analysis (TGA) curve of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention. The x-axis shows the temperature in degree Celsius (° C.), the y-axis shows the mass (loss) of the sample in weight percent (weight %).

FIG. 13: illustrates the NMR of N-(5-(5-((1R,2S)-2-fluorocyclopropyl)-1,2,4-oxadiazol-3-yl)-2-methylphenyl)imidazo[1,2-a]pyridine-3-carboxamide Form A obtained in d6-DMSO.

FIG. 14: illustrates the NMR of N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal.

### DETAILED DESCRIPTION OF THE INVENTION

### Crystalline Form

The present invention provides crystalline forms of N-(5-(5-((1R,2S)-2-fluorocyclopropyl)-1,2,4-oxadiazol-3-yl)-2-methylphenyl)imidazo[1,2-a]pyridine-3-carboxamide as the active pharmaceutical ingredient, in particular crystalline Form A of N-(5-(5-((1R,2S)-2-fluorocyclopropyl)-1,2,4-oxadiazol-3-yl)-2-methylphenyl)imidazo[1,2-a]pyridine-3-carboxamide and two hydrate forms of N-(5-(5-((1R,2S)-2-fluorocyclopropyl)-1,2,4-oxadiazol-3-yl)-2-methylphenyl)imidazo[1,2-a]pyridine-3-carboxamide; Form HA and Form HB.

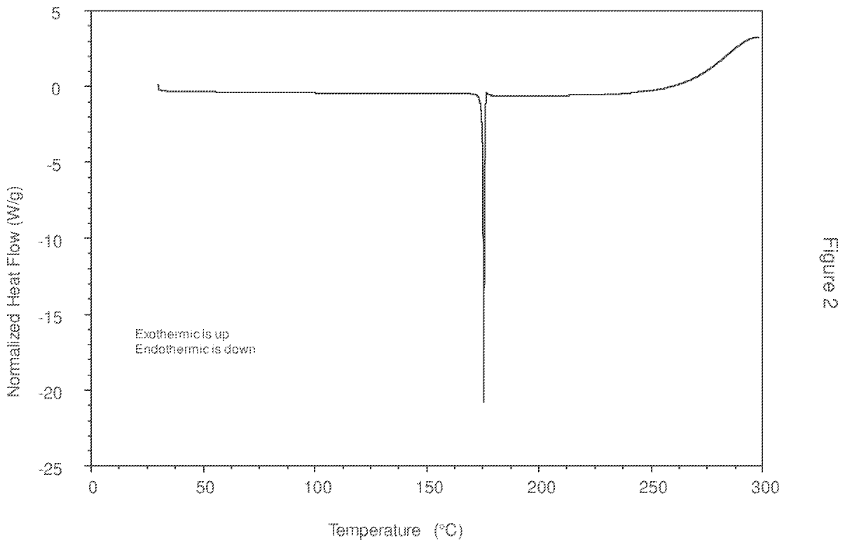

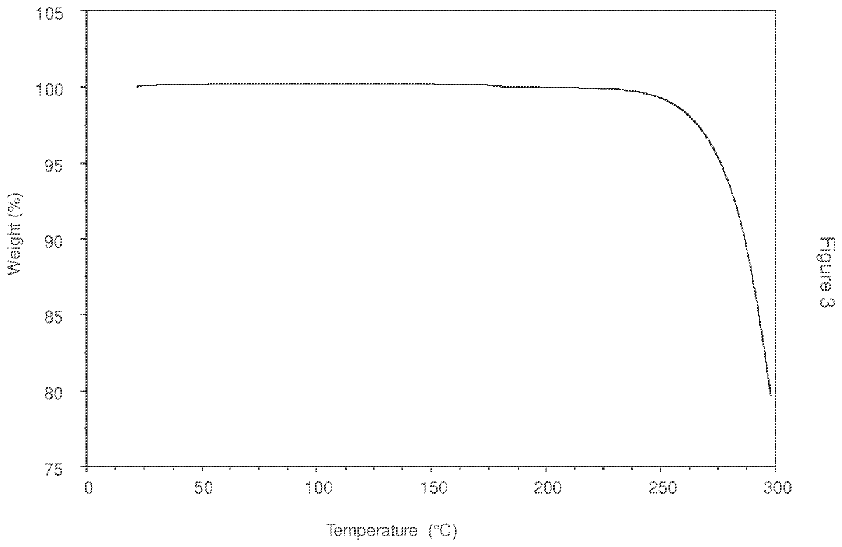

The N-(5-(5-((1R,2S)-2-fluorocyclopropyl)-1,2,4-oxadiazol-3-yl)-2-methylphenyl)imidazo[1,2-a]pyridine-3-carboxamide Form A of the present invention is physically stable toward temperature stress e.g. it shows no thermal events in a DSC experiment until it starts to melt at about 175° C. (FIG. 2). Moreover, a TGA experiment performed with the Form A of the present invention revealed no significant mass loss until melting (FIG. 3), which indicates the presence of an anhydrous and non-solvated solid-state form. In addition, the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention shows advantageous dissolution behaviour, good chemical stability e.g. against photodegradation and is characterized by excellent powder properties such as good flowability, high bulk density and good compressibility. All in all, these favorable attributes allow for a robust formulation and ensure a reliable safety and efficacy profile of a drug product containing Form A of N-(5-(5-((1R,2S)-2-fluorocyclopropyl)-1,2,4-oxadiazol-3-yl)-2-methylphenyl)imidazo[1,2-a]pyridine-3-carboxamide of the present invention during the whole shelf-life of the product.

The N-(5-(5-((1R,2S)-2-fluorocyclopropyl)-1,2,4-oxadiazol-3-yl)-2-methylphenyl)imidazo[1,2-a]pyridine-3-carboxamide Form A of the present invention may be characterized by analytical methods well known in the field of the pharmaceutical industry for characterizing crystalline solids. Such methods comprise but are not limited to powder and single X-ray diffraction, Fourier transform and Raman spectroscopy, DSC, TGA and GMS. The N-(5-(5-((1R,2S)-2-fluorocyclopropyl)-1,2,4-oxadiazol-3-yl)-2-methylphenyl)imidazo[1,2-a]pyridine-3-carboxamide Form HA of the present invention may be characterized by one of the aforementioned analytical methods or by combining two or more of them.

Both N-(5-(5-((1R,2S)-2-fluorocyclopropyl)-1,2,4-oxadiazol-3-yl)-2-methylphenyl)imidazo[1,2-a]pyridine-3-carboxamide Form HA and N-(5-(5-((1R,2S)-2-fluorocyclopropyl)-1,2,4-oxadiazol-3-yl)-2-methylphenyl)imidazo[1,2-a]pyridine-3-carboxamide Form HB of the present invention exhibit multiple thermal events under temperature stress. For example in a DSC experiment Form HA shows melting at about 87° C., melting at about 125° C. followed by recrystallization, melting at about 165° C. followed by recrystallization, and melting again with a final melting point of about 175° C. Similarly, a DSC experiment Form HB shows melting at about 110° C., followed by recrystallisation, melting at about 125° C. followed by recrystallization, melting at about 165° C. followed by recrystallization, and melting again with a final melting point of about 175° C. Moreover, a TGA experiment performed with the Form HA of the present invention revealed about 5% mass loss until melting, and Form HB revealed about a 4.5% mass loss until melting.

The N-(5-(5-((1R,2S)-2-fluorocyclopropyl)-1,2,4-oxadiazol-3-yl)-2-methylphenyl)imidazo[1,2-a]pyridine-3-carboxamide Form HA and Form HB of the present invention may be characterized by analytical methods well known in the field of the pharmaceutical industry for characterizing crystalline solids. Such methods comprise but are not limited to powder and single X-ray diffraction, Fourier transform and Raman spectroscopy, DSC, TGA and GMS. The crystalline forms HA and HB of the present invention may be characterized by one of the aforementioned analytical methods or by combining two or more of them.

The crystalline forms of the present invention may be characterized by any one of the following embodiments or by combining two or more of the following embodiments.

Embodiment 1: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, herein referred to as “Form A”, where N-(5-(5-((1R,2S)-2-fluorocyclopropyl)-1,2,4-oxadiazol-3-yl)-2-methylphenyl)imidazo[1,2-a]pyridine-3-carboxamide can be represented by the chemical structure as depicted in Formula A:

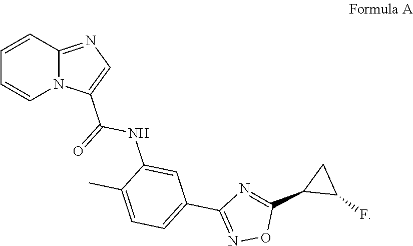 [SMILES: Cc1ccc(-c2noc([C@H]3C[C@@H]3F)n2)cc1NC(=O)c1cnc2ccccn12]

Embodiment 2: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, herein referred to as “Form HA”.

Embodiment 3: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, herein referred to as “Form HB”.

Embodiment 4: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of

(5.0±0.2) ° and (22.1±0.2)°; or

(5.0±0.2)°, (8.8±0.2)°, (17.4±0.2)°, (17.6±0.2)°, (22.1±0.2)° and (24.5±0.2)°; or

(5.0±0.2)°, (8.8±0.2)°, (15.2±0.2)°, (17.1±0.2)°, (17.4±0.2)°, (17.6±0.2)°, (22.1±0.2)° (22.8±0.2)° and (24.5±0.2)°; or

(5.0±0.2)°, (8.8±0.2) ° and (9.8±0.2)°; or

(5.0±0.2)°, (8.8±0.2)°, (9.8±0.2)° and (10.1±0.2)°; or

(5.0±0.2)°, (8.8±0.2)°, (9.8±0.2)°, (10.1±0.2) ° and (11.4±0.2)°; or

(5.0±0.2)°, (8.8±0.2)°, (9.8±0.2)°, (10.1±0.2)°, (11.4±0.2)° and (13.2±0.2)°; or

(5.0±0.2)°, (8.8±0.2)°, (9.8±0.2)°, (10.1±0.2)°, (11.4±0.2)°, (13.2±0.2)° and (15.2±0.2)°; or

(5.0±0.2)°, (8.8±0.2)°, (9.8±0.2)°, (10.1±0.2)°, (11.4±0.2)°, (13.2±0.2)°, (15.2±0.2)° and (17.1±0.2)° or (5.0±0.2)°, (8.8±0.2)°, (9.8±0.2)°, (10.1±0.2)°, (11.4±0.2)°, (13.2±0.2)°, (15.2±0.2)°, (17.1±0.2)° and (17.4±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)° and (17.6±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2) ° and (18.5±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2) ° and (19.7±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2)°, (19.7±0.2) ° and (20.3±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2)°, (19.7±0.2)°, (20.3±0.2) ° and (22.1±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2)°, (19.7±0.2)°, (20.3±0.2)°, (22.1±0.2) ° and (22.8±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2)°, (19.7±0.2)°, (20.3±0.2)°, (22.1±0.2)°, (22.8±0.2) ° and (24.5±0.2)°; or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2)°, (19.7±0.2)°, (20.3±0.2)°, (22.1±0.2)°, (22.8±0.2)°, (24.5±0.2) ° and (25.9±0.2); or

(5.0±0.2)°, (8.8±0.2)°, (9.8±0.2)°, (10.1±0.2)°, (11.4±0.2)°, (13.2±0.2)°, (15.2±0.2)°, (17.1±0.2)°, (17.4±0.2)°, (17.6±0.2)°, (18.5±0.2)°, (19.7±0.2)°, (20.3±0.2)°, (22.1±0.2)°, (22.8±0.2)°, (24.5±0.2)°, (25.9±0.2) ° and (26.7±0.2)°, when measured at RT with Cu-Kalpha radiation having a wavelength of 0.15406 nm.

Embodiment 5: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of (5.0±0.2) ° and (22.1±0.2)°, when measured at a temperature in the range of from 20 to 30° C. with Cu-Kalpha radiation having a wavelength of 0.15406 nm.

Embodiment 6: The crystalline form Embodiment 5 characterized by having a powder X-ray diffractogram comprising additional reflections at 2-Theta angles of (8.8±0.2)°, (17.4±0.2)°, (17.6±0.2)°, and (24.5±0.2)°, when measured at a temperature in the range of from 20 to 30° C. with Cu-Kalpha radiation having a wavelength of 0.15406 nm.

Embodiment 7: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of

(5.0±0.1) ° and (22.1±0.1)°; or

(5.0±0.1)°, (8.8±0.1)°, (17.4±0.1)°, (17.6±0.1)°, (22.1±0.1) ° and (24.5±0.1)°; or

(5.0±0.1)°, (8.8±0.1)°, (15.2±0.1)°, (17.1±0.1)°, (17.4±0.1)°, (17.6±0.1)°, (22.1±0.1)° (22.8±0.1) ° and (24.5±0.1)°; or

(5.0±0.1)°, (8.8±0.1) ° and (9.8±0.1)°; or

(5.0±0.1)°, (8.8±0.1)°, (9.8±0.1) ° and (10.1±0.1)°; or

(5.0±0.1)°, (8.8±0.1)°, (9.8±0.1)°, (10.1±0.1) ° and (11.4±0.1)°; or

(5.0±0.1)°, (8.8±0.1)°, (9.8±0.1)°, (10.1±0.1)°, (11.4±0.1)° and (13.2±0.1)°; or

(5.0±0.1)°, (8.8±0.1)°, (9.8±0.1)°, (10.1±0.1)°, (11.4±0.1)°, (13.2±0.1)° and (15.2±0.1)°; or

(5.0±0.1)°, (8.8±0.1)°, (9.8±0.1)°, (10.1±0.1)°, (11.4±0.1)°, (13.2±0.1)°, (15.2±0.1)° and (17.1±0.1) °; or

(5.0±0.1)°, (8.8±0.1)°, (9.8±0.1)°, (10.1±0.1)°, (11.4±0.1)°, (13.2±0.1)°, (15.2±0.1)°, (17.1±0.1)° and (17.4±0.1)°; or

(5.0±0.1)°, (8.8±0.1)°, (9.8±0.1)°, (10.1±0.1)°, (11.4±0.1)° (13.2±0.1)°, (15.2±0.1)°, (17.1±0.1)°, (17.4±0.1) ° and (17.6±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1) ° and (18.5±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)° and (19.7±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)°, (19.7±0.1)° and (20.3±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)°, (19.7±0.1)°, (20.3±0.1)° and (22.1±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)°, (19.7±0.1)°, (20.3±0.1)°, (22.1±0.1)° and (22.8±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)°, (19.7±0.1)°, (20.3±0.1)°, (22.1±0.1)°, (22.8±0.1)° and (24.5±0.1)°; or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)°, (19.7±0.1)°, (20.3±0.1)°, (22.1±0.1)°, (22.8±0.1)°. (24.5±0.1)° and (25.9±0.1); or

(5.0±0.1)°, (8.8±0.1)°, (9.8±0.1)°, (10.1±0.1)°, (11.4±0.1)°, (13.2±0.1)°, (15.2±0.1)°, (17.1±0.1)°, (17.4±0.1)°, (17.6±0.1)°, (18.5±0.1)°, (19.7±0.1)°, (20.3±0.1)°, (22.1±0.1)°, (22.8±0.1)°, (24.5±0.1)°, (25.9±0.1) ° and (26.7±0.1)°,

when measured at RT with Cu-Kalpha radiation having a wavelength of 0.15406 nm.

Embodiment 8: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of (5.0±0.1) ° and (22.1±0.1)°, when measured at a temperature in the range of from 20 to 30° C. with Cu-Kalpha radiation having a wavelength of 0.15406 nm.

Embodiment 9: The crystalline form Embodiment 8 characterized by having a powder X-ray diffractogram comprising additional reflections at 2-Theta angles of (8.8±0.1)°, (17.4±0.1)°, (17.6±0.1)° and (24.5±0.1)°, when measured at a temperature in the range of from 20 to 30° C. with Cu-Kalpha radiation having a wavelength of 0.15406 nm.

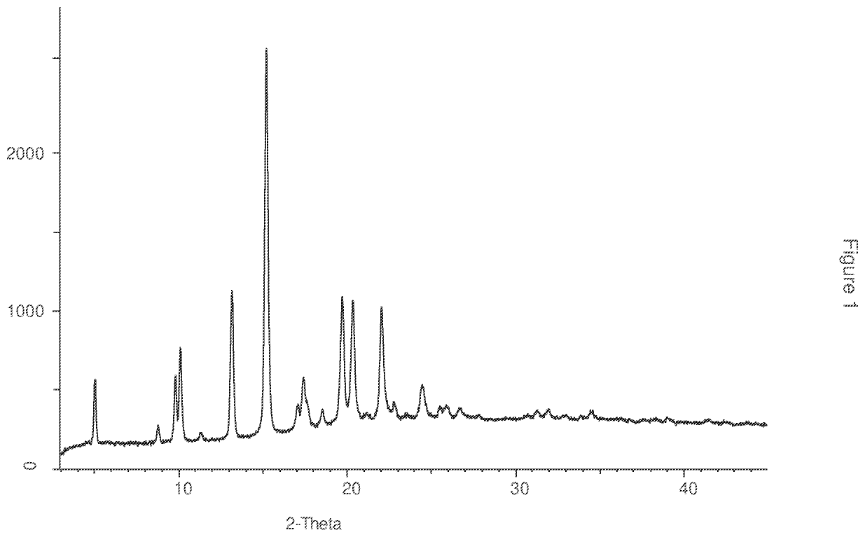

Embodiment 10: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram essentially the same as shown in FIG. 1 of the present invention, when measured at room temperature with Cu-Kalpha radiation having a wavelength of 0.15406 nm.

Embodiment 11: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized in that the crystalline form is anhydrous.

Embodiment 12: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized in that the crystalline form is non-solvated.

Embodiment 13: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a differential scanning calorimetry curve comprising an endothermic peak having an onset temperature of (175.0±0.5) ° C., when measured at a heating rate of 10 K/min.

Embodiment 14: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a differential scanning calorimetry curve comprising an endothermic peak having a peak temperature of (175.2±0.5)° C., when measured at a heating rate of 10 K/min.

Embodiment 15: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve showing a mass loss of 0.01 weight % or less, preferably of 0.007 weight % or less based on the weight of the crystalline form, when heated from room temperature to 180° C. at a rate of 10 K/min.

Embodiment 16: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve showing a mass loss of not more than 0.01 weight %, based on the weight of the crystalline form, when heated from 30° C. to 180° C. at a rate of 10 K/min.

Embodiment 17: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of:

(8.0±0.2)° and (32.6±0.2)°; or

(6.4±0.2)°, (8.0±0.2)° and (10.1±0.2)°; or

(6.4±0.2)°, (8.0±0.2)°, (10.1±0.2) ° and (10.7±0.2)°; or

(6.4±0.2)°, (8.0±0.2)°, (10.1±0.2)°, (10.7±0.2) ° and (12.8±0.2)°; or

(6.4±0.2)°, (8.0±0.2)°, (10.1±0.2)°, (10.7±0.2)°. (12.8±0.2) ° and (13.6±0.2)°;

(6.4±0.2)°, (8.0±0.2)°, (10.1±0.2)°, (10.7±0.2)°. (12.8±0.2)°, (13.6±0.2) ° and (16.3±0.2)°; or

(6.4±0.2)°, (8.0±0.2)°, (10.1±0.2)°, (10.7±0.2)°, (12.8±0.2)°, (13.6±0.2)°, (16.3±0.2)° and (16.8±0.2)°; or

(6.4±0.2)°, (8.0±0.2)°, (10.1±0.2)°, (10.7±0.2)°, (12.8±0.2)°, (13.6±0.2)°, (16.3±0.2)°, (16.8±0.2)° and (18.4±0.2)°;

(6.4±0.2)°, (8.0±0.2)°, (10.1±0.2)°, (10.7±0.2)°, (12.8±0.2)°, (13.6±0.2)°, (16.3±0.2)°, (16.8±0.2)°, (18.4±0.2)° and (19.3±0.2)°; or

(6.4±0.2)°, (8.0±0.2)°, (10.1±0.2)°, (10.7±0.2)°, (12.8±0.2)°, (13.6±0.2)°, (16.3±0.2)°, (16.8±0.2)°, (18.4±0.2)°, (19.3±0.2) ° and (19.9±0.2)°; or

(6.4±0.2)°, (8.0±0.2)°, (10.1±0.2)°, (10.7±0.2)°, (12.8±0.2)°, (13.6±0.2)°, (16.3±0.2)°, (16.8±0.2)°, (18.4±0.2)°, (19.3±0.2)°, (19.9±0.2) ° and (21.6±0.2)°; or

(6.4±0.2)°, (8.0±0.2)°, (10.1±0.2)°, (10.7±0.2)°, (12.8±0.2)°, (13.6±0.2)°, (16.3±0.2)°, (16.8±0.2)°, (18.4±0.2)°. (19.3±0.2)°, (19.9±0.2)°, (21.6±0.2) ° and (25.9±0.2)°; or

(6.4±0.2)°, (8.0±0.2)°, (10.1±0.2)°, (10.7±0.2)°, (12.8±0.2)°, (13.6±0.2)°, (16.3±0.2)°, (16.8±0.2)°, (18.4±0.2)°, (19.3±0.2)°, (19.9±0.2)°, (21.6±0.2)°, (25.9±0.2)° and (26.9±0.2)°; or

(6.4±0.2)°, (8.0±0.2)°, (10.1±0.2)°, (10.7±0.2)°, (12.8±0.2)°, (13.6±0.2)°, (16.3±0.2)°, (16.8±0.2)°, (18.4±0.2)°, (19.3±0.2)°, (19.9±0.2)°, (21.6±0.2)°, (25.9±0.2)°, (26.9±0.2)° and (32.6±0.2)°,

when measured at RT with Cu-Kalpha radiation having a wavelength of 0.15406 nm.

Embodiment 18: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of (8.0±0.2) ° and (32.6±0.2)°; when measured at RT with Cu-Kalpha radiation having a wavelength of 0.15406 nm.

Embodiment 19: The crystalline form Embodiment 18 characterized by having a powder X-ray diffractogram comprising additional reflections at 2-Theta angles of (12.8±0.2)°, (21.6±0.2) ° and (25.9±0.2)°, when measured at a temperature in the range of from 20 to 30° C. with Cu-Kalpha radiation having a wavelength of 0.15406 nm.

Embodiment 20: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of:

(8.0±0.1) ° and (32.6±0.1)°; or

(6.4±0.1)°, (8.0±0.1)° and (10.1±0.1)°; or

(6.4±0.1)°, (8.0±0.1)°, (10.1±0.1) ° and (10.7±0.1)°; or

(6.4±0.1)°, (8.0±0.1)°, (10.1±0.1)°, (10.7±0.1) ° and (12.8±0.1)°; or

(6.4±0.1)°, (8.0±0.1)°, (10.1±0.1)°, (10.7±0.1)°, (12.8±0.1) ° and (13.6±0.1)°;

(6.4±0.1)°, (8.0±0.1)°, (10.1±0.1)°, (10.7±0.1)°, (12.8±0.1)°, (13.6±0.1) ° and (16.3±0.1)°; or

(6.4±0.1)°, (8.0±0.1)°, (10.1±0.1)°, (10.7±0.1)°, (12.8±0.1)°, (13.6±0.1)°, (16.3±0.1)° and (16.8±0.1)°; or

(6.4±0.1)°, (8.0±0.1)°, (10.1±0.1)°, (10.7±0.1)°, (12.8±0.1)°, (13.6±0.1)°, (16.3±0.1)°, (16.8±0.1) ° and (18.4±0.1)°;

(6.4±0.1)°, (8.0±0.1)°, (10.1±0.1)°, (10.7±0.1)°, (12.8±0.1)°, (13.6±0.1)°, (16.3±0.1)°, (16.8±0.1)°, (18.4±0.1)° and (19.3±0.1)°; or

(6.4±0.1)°, (8.0±0.1)°, (10.1±0.1)°, (10.7±0.1)°, (12.8±0.1)°, (13.6±0.1)°, (16.3±0.1)°, (16.8±0.1)°, (18.4±0.1)° (19.3±0.1)° and (19.9±0.1)°: or

(6.4±0.1)°, (8.0±0.1)°, (10.1±0.1)°, (10.7±0.1)°, (12.8±0.1)°, (13.6±0.1)°, (16.3±0.1)°, (16.8±0.1)°, (18.4±0.1)°, (19.3±0.1)°, (19.9±0.1)° and (21.6±0.1)°; or

(6.4±0.1)°, (8.0±0.1)°, (10.1±0.1)°, (10.7±0.1)°, (12.8±0.1)°, (13.6±0.1)°, (16.3±0.1)°, (16.8±0.1)°, (18.4±0.1)°, (19.3±0.1)°, (19.9±0.1)°, (21.6±0.1)° and (25.9±0.1)°; or

(6.4±0.1)°, (8.0±0.1)°, (10.1±0.1)°, (10.7±0.1)°, (12.8±0.1)°, (13.6±0.1)°, (16.3±0.1)°, (16.8±0.1)°, (18.4±0.1)°, (19.3±0.1)°, (19.9±0.1)°, (21.6±0.1)°, (25.9±0.1)° and (26.9±0.1)°; or

(6.4±0.1)°, (8.0±0.1)°, (10.1±0.1)°, (10.7±0.1)°, (12.8±0.1)°, (13.6±0.1)°, (16.3±0.1)°, (16.8±0.1)°, (18.4±0.1)°, (19.3±0.1)°, (19.9±0.1)°, (21.6±0.1)°, (25.9±0.1)°, (26.9±0.1) ° and (32.6±0.1)°,

when measured at RT with Cu-Kalpha radiation having a wavelength of 0.15406 nm.

Embodiment 21: A crystalline form of N-(5-(5-((1R,2S)-2-fluorocyclopropyl)-1,2,4-oxadiazol-3-yl)-2-methylphenyl)imidazo[1,2-a]pyridine-3, carboxamide, characterized by having a powder X-ray diffractogram comprising reflections at 2-Theta angles of (8.0±0.1) ° and (32.6 15±0.1)°; when measured at RT with Cu-Kalpha radiation having a wavelength of 0.15406 nm.

Embodiment 22: The crystalline form Embodiment 21 characterized by having a powder X-ray diffractogram comprising additional reflections at 2-Theta angles of (12.8±0.1)°, (21.6±0.1) ° and (25.9±0.1)°, when measured at a temperature in the range of from 20 to 30° C. with Cu-Kalpha radiation having a wavelength of 0.15406 nm.

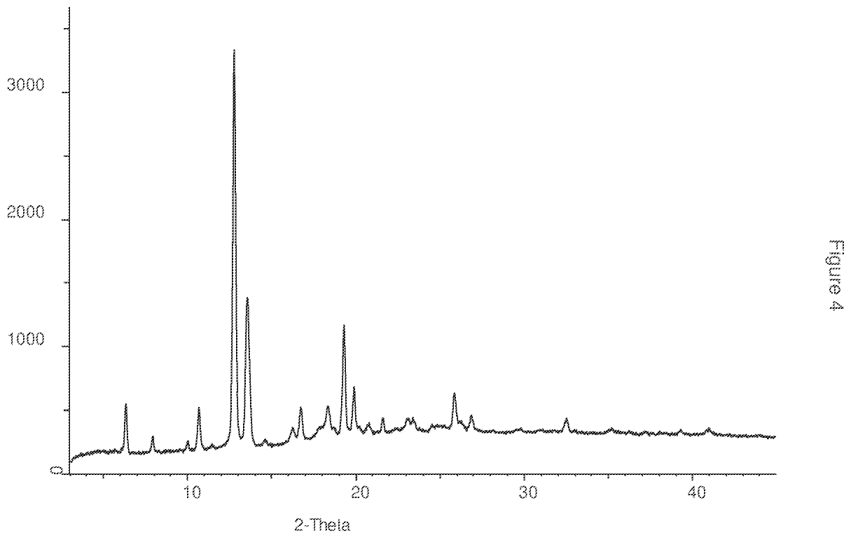

Embodiment 23: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram essentially the same as shown in FIG. 4 of the present invention, when measured at room temperature with Cu-Kalpha radiation having a wavelength of 0.15406 nm.

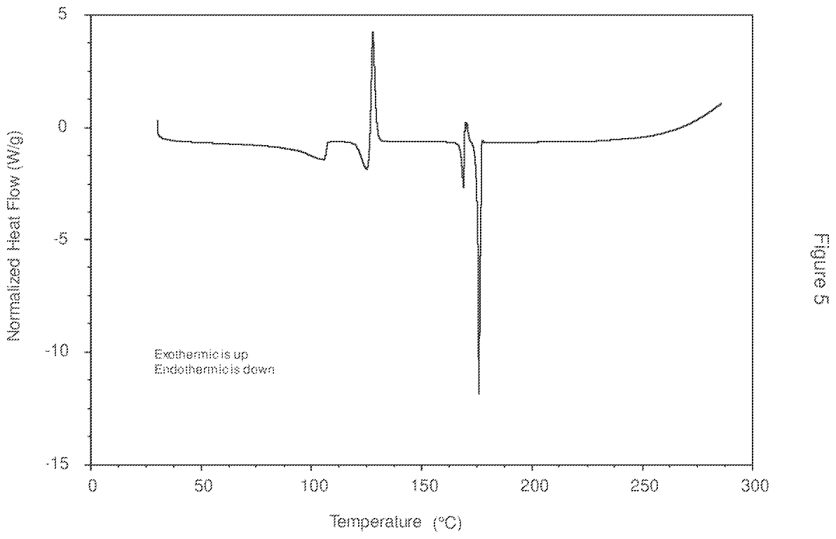

Embodiment 24: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a differential scanning calorimetry curve essentially the same as shown in FIG. 5 when measured with a DSC at a heating rate of 10 K/min.

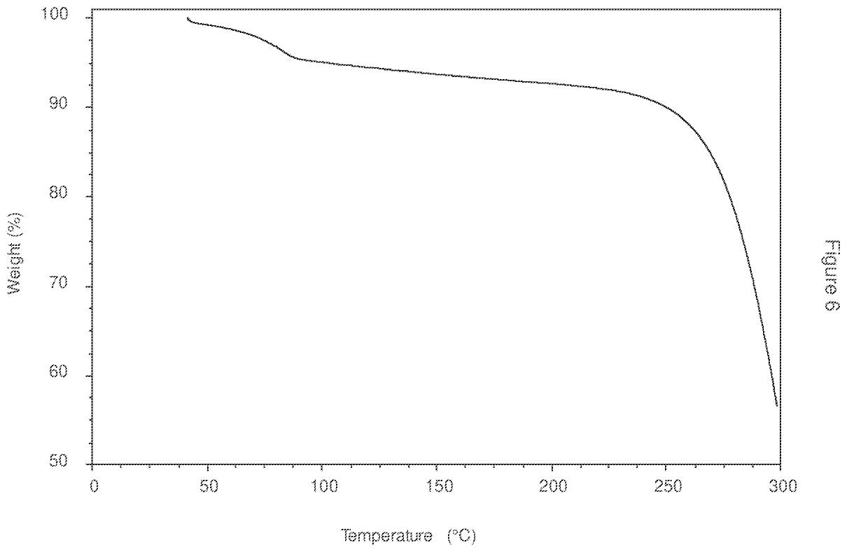

Embodiment 25: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve essentially the same as shown in FIG. 6 when heated from room temperature to 112° C. at a rate of 10 K/min.

Embodiment 26: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve showing a mass loss of 5.5 weight % or less, preferably of 5.3 weight % or less based on the weight of the crystalline form, when heated from room temperature to 112° C. at a rate of 10 K/min.

Embodiment 27: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve showing a mass loss of 5.5 weight % or less, preferably of 5.3 weight % or less based on the weight of the crystalline form, when heated from 30° C. to 112° C. at a rate of 10 K/min.

Embodiment 28: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of:

(23.8±0.2) ° and (29.7±0.2)°; or

(23.5±0.2)°, (23.8±0.2) ° and (28.7±0.2)°; or

(6.7±0.2)°, (10.1±0.2) ° and (10.7±0.2)°; or

(6.7±0.2)°, (10.1±0.2)°, (10.7±0.2) ° and (11.2±0.2)°; or

(6.7±0.2)°, (10.1±0.2)°, (10.7±0.2)°, (11.2±0.2) ° and (13.6±0.2)°: or (6.7±0.2)°, (10.1±0.2)°, (10.7±0.2)°, (11.2±0.2)°, (13.6±0.2) ° and (16.5±0.2)°; or

(6.7±0.2)°, (10.1±0.2)°, (10.7±0.2)°, (11.2±0.2)°, (13.6±0.2)°, (16.5±0.2) ° and (18.0±0.2)°; or

(6.7±0.2)°, (10.1±0.2)°, (10.7±0.2)°, (11.2±0.2)°, (13.6±0.2)°, (16.5±0.2)°, (18.0±0.2)° and (19.1±0.2)°; or

(6.7±0.2)°, (10.1±0.2)°, (10.7±0.2)°, (11.2±0.2)°, (13.6±0.2)°, (16.5±0.2)°, (18.0±0.2)°, (19.1±0.2) ° and (20.2±0.2)°; or

(6.7±0.2)°, (10.1±0.2)°, (10.7±0.2)°, (11.2±0.2)°, (13.6±0.2)°, (16.5±0.2)°, (18.0±0.2)°, (19.1±0.2)°, (20.2±0.2) ° and (23.5±0.2)°: or (6.7±0.2)°, (10.1±0.2)°, (10.7±0.2)°, (11.2±0.2)°, (13.6±0.2)°, (16.5±0.2)°, (18.0±0.2)°, (19.1±0.2)°, (20.2±0.2)°, (23.5±0.2)° and (23.8±0.2)°; or

(6.7±0.2)°, (10.1±0.2)°, (10.7±0.2)°, (11.2±0.2)°, (13.6±0.2)°, (16.5±0.2)°, (18.0±0.2)°, (19.1±0.2)°, (20.2±0.2)°, (23.5±0.2)°, (23.8±0.2)° and (25.0±0.2)°;

(6.7±0.2)°, (10.1±0.2)°, (10.7±0.2)°, (11.2±0.2)°, (13.6±0.2)°, (16.5±0.2)°, (18.0±0.2)°, (19.1±0.2)°, (20.2±0.2)°, (23.5±0.2)°, (23.8±0.2)°, (25.0±0.2)° and (26.4±0.2)°; or

(6.7±0.2)°, (10.1±0.2)°, (10.7±0.2)°, (11.2±0.2)°, (13.6±0.2)°, (16.5±0.2)°, (18.0±0.2)°, (19.1±0.2)°, (20.2±0.2)°, (23.5±0.2)°, (23.8±0.2)°, (25.0±0.2)°, (26.4±0.2) ° and (28.7±0.2)°; or

(6.7±0.2)°, (10.1±0.2)°, (10.7±0.2)°, (11.2±0.2)°, (13.6±0.2)°, (16.5±0.2)°, (18.0±0.2)°, (19.1±0.2)°, (20.2±0.2)°, (23.5±0.2)°, (23.8±0.2)°, (25.0±0.2)°, (26.4±0.2)°, (28.7±0.2) ° and (29.7±0.2)°,

when measured at RT with Cu-Kalpha radiation having a wavelength of 0.15406 nm.

Embodiment 29: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of (23.8±0.2)° and (29.7±0.2)°, when measured at RT with Cu-Kalpha radiation having a wavelength of 0.15406 nm.

Embodiment 30: The crystalline form Embodiment 18 characterized by having a powder X-ray diffractogram comprising additional reflections at 2-Theta angles of (23.5±0.2)°, (23.8±0.2)° and (28.7±0.2)°, when measured at a temperature in the range of from 20 to 30° C. with Cu-Kalpha radiation having a wavelength of 0.15406 nm.

Embodiment 31: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, Form HB, characterized by having a powder X-ray diffractogram comprising reflections at 2-Theta angles of:

(23.8±0.1) ° and (29.7±0.1)°; or

(23.5±0.1)°, (23.8±0.1) ° and (28.7±0.1)°; or

(6.7±0.1)°, (10.1±0.1) ° and (10.7±0.1)°; or

(6.7±0.1)°, (10.1±0.1)°, (10.7±0.1) ° and (11.2±0.1)°; or

(6.7±0.1)°, (10.1±0.1)°, (10.7±0.1)°, (11.2±0.1) ° and (13.6±0.1)°; or

(6.7±0.1)°, (10.1±0.1)°, (10.7±0.1)°, (11.2±0.1)°, (13.6±0.1)° and (16.5±0.1)°; or

(6.7±0.1)°, (10.1±0.1)°, (10.7±0.1)°, (11.2±0.1)°, (13.6±0.1)°, (16.5±0.1) ° and (18.0±0.1)°; or

(6.7±0.1)°, (10.1±0.1)°, (10.7±0.1)°, (11.2±0.1)°, (13.6±0.1)°, (16.5±0.1)°, (18.0±0.1)° and (19.1±0.1)°; or

(6.7±0.1)°, (10.1±0.1)°, (10.7±0.1)°, (11.2±0.1)°, (13.6±0.1)°, (16.5±0.1)°, (18.0±0.1)°, (19.1±0.1)° and (20.1±0.1)°; or

(6.7±0.1)°, (10.1±0.1)°, (10.7±0.1)°, (11.2±0.1)°, (13.6±0.1)°, (16.5±0.1)°, (18.0±0.1)°, (19.1±0.1)°, (20.1±0.1)° and (23.5±0.1)°; or

(6.7±0.1)°, (10.1±0.1)°, (10.7±0.1)°, (11.2±0.1)°, (13.6±0.1)°, (16.5±0.1)°, (18.0±0.1)°, (19.1±0.1)°, (20.1±0.1)°, (23.5±0.1)° and (23.8±0.1)°; or

(6.7±0.1)°, (10.1±0.1)°, (10.7±0.1)°, (11.2±0.1)°, (13.6±0.1)°, (16.5±0.1)°, (18.0±0.1)°, (19.1±0.1)°, (20.1±0.1)°, (23.5±0.1)°, (23.8±0.1) ° and (25.0±0.1)°;

(6.7±0.1)°, (10.1±0.1)°, (10.7±0.1)°, (11.2±0.1)°, (13.6±0.1)°, (16.5±0.1)°, (18.0±0.1)°, (19.1±0.1)°, (20.1±0.1)°, (23.5±0.1)°, (23.8±0.1)°, (25.0±0.1) ° and (26.4±0.1)°; or

(6.7±0.1)°, (10.1±0.1)°, (10.7±0.1)°, (11.2±0.1)°, (13.6±0.1)°, (16.5±0.1)°, (18.0±0.1)°, (19.1±0.1)°, (20.1±0.1)°, (23.5±0.1)°, (23.8±0.1)°, (25.0±0.1)°, (26.4±0.1) ° and (28.7±0.1)°; or

(6.7±0.1)°, (10.1±0.1)°, (10.7±0.1)°, (11.2±0.1)°, (13.6±0.1)°, (16.5±0.1)°, (18.0±0.1)°, (19.1±0.1)°, (20.2±0.1)°, (23.5±0.1)°, (23.8±0.1)°, (25.0±0.1)°, (26.4±0.1)°, (28.7±0.1) ° and (29.7±0.1)°,

when measured at RT with Cu-Kalpha radiation having a wavelength of 0.15406 nm.

Embodiment 32: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram comprising reflections at 2-Theta angles of (23.8±0.1) ° and (29.7±0.1)°, when measured at RT with Cu-Kalpha radiation having a wavelength of 0.15406 nm.

Embodiment 33: The crystalline form Embodiment 18 characterized by having a powder X-ray diffractogram comprising additional reflections at 2-Theta angles of (23.5±0.1)°, (23.8±0.1) ° and (28.7±0.1)°, when measured at a temperature in the range of from 20 to 30° C. with Cu-Kalpha radiation having a wavelength of 0.15406 nm.

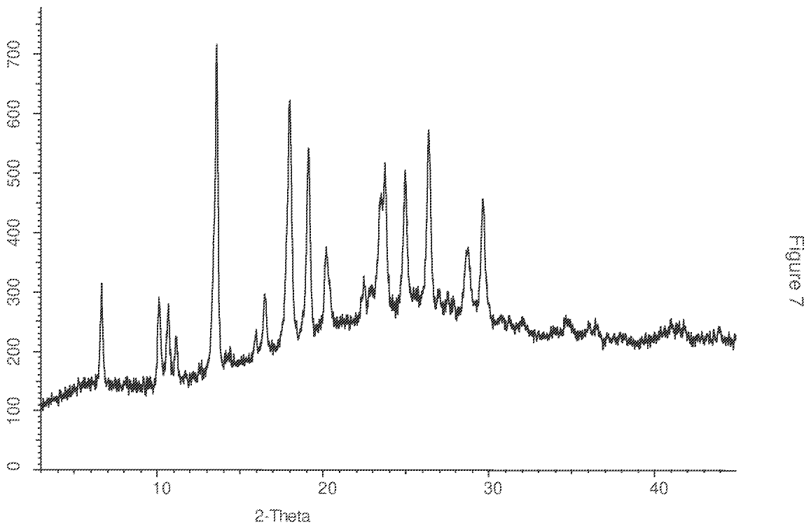

Embodiment 34: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a powder X-ray diffractogram essentially the same as shown in FIG. 7 of the present invention, when measured at room temperature with Cu-Kalpha radiation having a wavelength of 0.15406 nm.

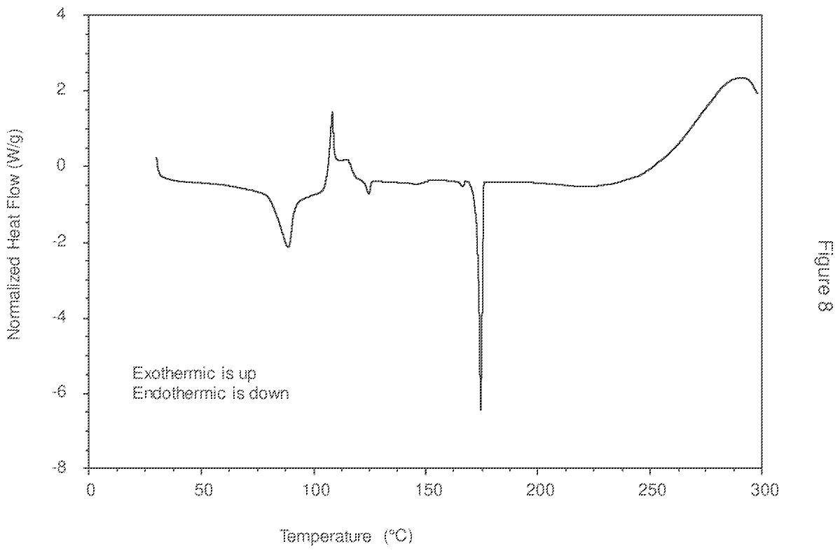

Embodiment 35: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a differential scanning calorimetry curve essentially the same as shown in FIG. 8 when measured with DSC at a heating rate of 10 K/min.

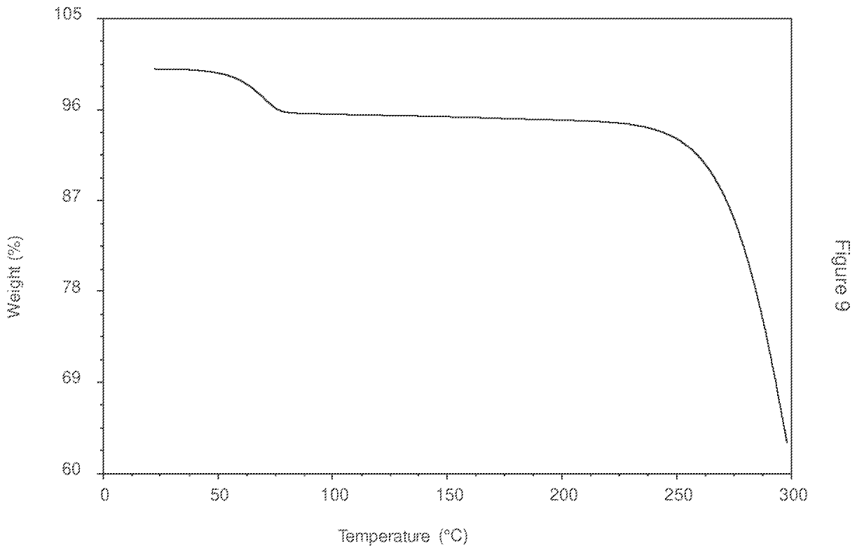

Embodiment 36: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve essentially the same as shown in FIG. 9 when heated from room temperature to 100° C. at a rate of 10 K/min.

Embodiment 37: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve showing a mass loss of 5.0 weight % or less, preferably of 4.5 weight % or less based on the weight of the crystalline form, when heated from room temperature to 100° C. at a rate of 10 K/min.

Embodiment 38: A crystalline form of N-(5-(5-((1R,2S)-2-fluorocyclopropyl)-1,2,4-oxadiazol-3-yl)-2-methylphenyl)imidazo[1,2-a]pyridine-3-carboxamide, characterized by having a thermogravimetric analysis curve showing a mass loss of 5.0 weight % or less, preferably of 4.5 weight % or less based on the weight of the crystalline form, when heated from 30° C. to 100° C. at a rate of 10 K/min.

Thermal analyses such as DSC and TGA revealed that the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention is thermally highly stable e.g. does not undergo phase transformations or decomposition until it melts at about 175° C.

This is in contrast to the Form HA and Form HB of free form N-(5-(5-((1R,2S)-2-fluorocyclopropyl)-1,2,4-oxadiazol-3-yl)-2-methylphenyl)imidazo[1,2-a]pyridine-3-carboxamide, which show thermal events such as dehydration/desolvation and recrystallization events during DSC experiments, indicating solvent/water losses and phase transformations. It is worth mentioning that Form A and Form B of N-(5-(5-((1R,2S)-2-fluorocyclopropyl)-1,2,4-oxadiazol-3-yl)-2-methylphenyl)imidazo[1,2-a]pyridine-3-carboxamide appear to at least partially transform to N-(5-(5-((1R,2S)-2-fluorocyclopropyl)-1,2,4-oxadiazol-3-yl)-2-methylphenyl)imidazo[1,2-a]pyridine-3-carboxamide Form A during DSC experiments, which is indicated by the final melting endotherm having a peak temperature of about 175° C., which can be assigned to the melting of N-(5(5((1R,2S)-2-fluorocyclopropyl)-1,2,4-oxadiazol-3-yl)-2-methylphenyl)imidazo[1,2-a]pyridine-3-carboxamide Form A.

Hence, the thermal stability of the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention is superior compared to the hydrated Forms HA and HB of N-(5-(5-((1R,2S)-2-fluorocyclopropyl)-1,2,4-oxadiazol-3-yl)-2-methylphenyl)imidazo[1,2-a]pyridine-3-carboxamide.

Co-Crystal The present invention provides a pharmaceutical co-crystal composed of N-(5-(5-((1R,2S)-2-fluorocyclopropyl)-1,2,4-oxadiazol-3-yl)-2-methylphenyl)imidazo[1,2-a]pyridine-3-carboxamide as the active pharmaceutical ingredient and fumaric acid as the co-crystal former.

The N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention is physically stable toward temperature stress e.g. it shows no thermal events in a DSC experiment until it starts to melt at about 229° C. Moreover, a TGA experiment performed with the co-crystal of the present invention revealed no significant mass loss until melting, which indicates the presence of an anhydrous and non-solvated solid-state form. In addition, the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention shows advantageous dissolution behavioiur, good chemical stability e.g. against photodegradation and is characterized by excellent powder properties such as good flowability, high bulk density and good compressibility. All in all, these favorable attributes allow for a robust formulation and ensure a reliable safety and efficacy profile of a drug product containing the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention during the whole shelf-life of the product.

The N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention may be characterized by analytical methods well known in the field of the pharmaceutical industry for characterizing crystalline solids. Such methods comprise but are not limited to powder and single X-ray diffraction, Fourier transform and Raman spectroscopy, DSC, TGA and GMS. The co-crystal of the present invention may be characterized by one of the aforementioned analytical methods or by combining two or more of them. In particular, the co-crystal of the present invention may be characterized by any one of the following embodiments or by combining two or more of the following embodiments.

Embodiment 39: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid.

Embodiment 40: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid characterized by having the chemical structure as depicted in Formula B

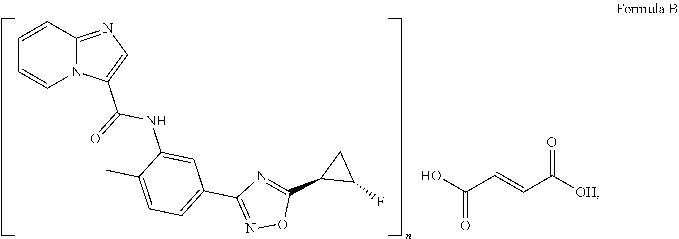

wherein n is in the range of from 1.8 to 2.2, preferably of from 1.9 to 2.1, even more preferably of from 1.95 to 2.05 and most preferably n is 2.0.

Embodiment 41: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is in the range of from 1.8 to 2.2:1.

Embodiment 42: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is in the range of from preferably of from 1.9 to 2.1:1.

Embodiment 43: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is in the range of from even more preferably of from 1.95 to 2.05:1.

Embodiment 44: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is in the range of from and most preferably 2:1.

Embodiment 45: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid characterized by having a powder X-ray diffractogram (PXRD) comprising reflections at 2-Theta angles of:

(12.3±0.2) ° and (27.3±0.2)°; or

(4.9±0.2)°, (10.0±0.2) ° and (11.5±0.2)°; or

(4.9±0.2)°, (10.0±0.2)°, (11.5±0.2) ° and (12.3±0.2)°; or

(4.9±0.2)°, (10.0±0.2)°, (11.5±0.2)°, (12.3±0.2)° and (14.9±0.2)°; or

(4.9±0.2)°, (10.0±0.2)°, (11.5±0.2)°, (12.3±0.2)°, (14.9±0.2) ° and (15.6±0.2)°; or

(4.9±0.2)°, (10.0±0.2)°, (11.5±0.2)°, (12.3±0.2)°, (14.9±0.2)°, (15.6±0.2) ° and (16.5±0.2)°; or

(4.9±0.2)°, (10.0±0.2)°, (11.5±0.2)°, (12.3±0.2)°, (14.9±0.2)°, (15.6±0.2)°, (16.5±0.2)° and (18.6±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2) ° and (20.1±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2)°, (20.1±0.2) ° and (21.2±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2)°, (20.1±0.2)°, (21.2±0.2) ° and (22.6±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2)°, (20.1±0.2)°, (21.2±0.2)°, (22.6±0.2) ° and (22.8±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2)°, (20.1±0.2)°, (21.2±0.2)°, (22.6±0.2)°, (22.8±0.2) ° and (25.4±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2)°, (20.1±0.2)°, (21.2±0.2)°, (22.6±0.2)°, (22.8±0.2)°, (25.4±0.2) ° and (26.5±0.2)°; or

(4.9±0.2)°, (10.0±0.2)°, (11.5±0.2)°, (12.3±0.2)°, (14.9±0.2)°, (15.6±0.2)°, (16.5±0.2)°, (18.6±0.2)°, (20.1±0.2)°, (21.2±0.2)°, (22.6±0.2)°, (22.8±0.2)°, (25.4±0.2)°, (26.5±0.2) ° and (27.3±0.2)°,

when measured at RT with Cu-Kalpha radiation having a wavelength of 0.15406 nm.

Embodiment 46: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid characterized by having a powder X-ray diffractogram (PXRD) comprising reflections at 2-Theta angles of (12.3±0.2) ° and (27.3±0.2)°, when measured at a temperature in the range of from 20 to 30° C. with Cu-Kalpha radiation having a wavelength of 0.15406 nm.

Embodiment 47: The co-crystal of Embodiment 8 characterized by having a powder X-ray diffractogram (PXRD) comprising additional reflections at 2-Theta angles of (14.9.0±0.2)°, (16.5±0.2)°, (21.2±0.2) ° and (25.4±0.2)°, when measured at a temperature in the range of from 20 to 30° C. with Cu-Kalpha radiation having a wavelength of 0.15406 nm.

Embodiment 48: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with Fumaric acid characterized by having a powder X-ray diffractogram (PXRD) comprising reflections at 2-Theta angles of:

(12.3±0.1) ° and (27.3±0.1)°; or

(4.9±0.1)°, (10.0±0.1) ° and (11.5±0.1)°; or

(4.9±0.1)°, (10.0±0.1)°, (11.5±0.1) ° and (12.3±0.1)°; or

(4.9±0.1)°, (10.0±0.1)°, (11.5±0.1)°, (12.3±0.1)° and (14.9±0.1)°; or

(4.9±0.1)°, (10.0±0.1)°, (11.5±0.1)°, (12.3±0.1)°, (14.9±0.1) ° and (15.6±0.1)°; or

(4.9±0.1)°, (10.0±0.1)°, (11.5±0.1)°, (12.3±0.1)°, (14.9±0.1)°, (15.6±0.1) ° and (16.5±0.1)°; or

(4.9±0.1)°, (10.0±0.1)°, (11.5±0.1)°, (12.3±0.1)°, (14.9±0.1)°, (15.6±0.1)°, (16.5±0.1)° and (18.6±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1) ° and (20.1±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1)°, (20.1±0.1) ° and (21.2±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1)°, (20.1±0.1)°, (21.2±0.1) ° and (22.6±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1)°, (20.1±0.1)°, (21.2±0.1)°, (22.6±0.1) ° and (22.8±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1)°, (20.1±0.1)°, (21.2±0.1)°, (22.6±0.1)°, (22.8±0.1) ° and (25.4±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1)°, (20.1±0.1)°, (21.2±0.1)°, (22.6±0.1)°, (22.8±0.1)°, (25.4±0.1)° and (26.5±0.1)°; or

(4.9±0.1)°, (10.0±0.1)°, (11.5±0.1)°, (12.3±0.1)°, (14.9±0.1)°, (15.6±0.1)°, (16.5±0.1)°, (18.6±0.1)°, (20.1±0.1)°, (21.2±0.1)°, (22.6±0.1)°, (22.8±0.1)°, (25.4±0.1)°, (26.5±0.1) ° and (27.3±0.1)°,

when measured at RT with Cu-Kalpha radiation having a wavelength of 0.15406 nm.

Embodiment 49: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid characterized by having a powder X-ray diffractogram (PXRD) comprising reflections at 2-Theta angles of (12.3±0.1) ° and (27.3±0.1)°, when measured at a temperature in the range of from 20 to 30° C. with Cu-Kalpha radiation having a wavelength of 0.15406 nm.

Embodiment 50: The co-crystal of Embodiment 11 characterized by having a powder X-ray diffractogram (PXRD) comprising additional reflections at 2-Theta angles of (14.9.0±0.1)°, (16.5±0.1)°, (21.2±0.1) ° and (25.4±0.1)°, when measured at a temperature in the range of from 20 to 30° C. with Cu-Kalpha radiation having a wavelength of 0.15406 nm.

Embodiment 51: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid, characterized by having a powder X-ray diffractogram (PXRD) essentially the same as shown in FIG. 1 of the present invention, when measured at room temperature with Cu-Kalpha radiation having a wavelength of 0.15406 nm.

Embodiment 52: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid characterized in that the co-crystal is anhydrous.

Embodiment 53: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid characterized in that the co-crystal is non-solvated.

Embodiment 54: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid, characterized by having a differential scanning calorimetry (DSC) curve comprising an endothermic peak, preferably a single endothermic peak, having an onset temperature of (227±1)° C., when measured with DSC at a heating rate of 10 K/min.

Embodiment 55: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid, characterized by having a differential scanning calorimetry (DSC) curve comprising an endothermic peak, preferably a single endothermic peak, having a peak temperature of (229±1) ° C., when measured with DSC at a heating rate of 10 K/min.

Embodiment 56: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid, characterized by having a thermogravimetric analysis (TGA) curve showing a mass loss of 1.5 weight % or less, preferably of 1.4 weight % or less based on the weight of the co-crystal, when heated from RT to 150° C. at a rate of 10 K/min.

Embodiment 57: A co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid, characterized by having a thermogravimetric analysis (TGA) curve showing a mass loss of 2.5 weight % or less, preferably of 2.0 weight % or less based on the weight of the co-crystal, when heated from RT to 200° C. at a rate of 10 K/min.

Thermal analyses such as DSC and TGA revealed that the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention is thermally highly stable e.g. does not undergo phase transformations or decomposition until it melts at about 229° C.

This is in contrast to the Form A and Form B of N-(5-(5-((1R,2S)-2-fluorocyclopropyl)-1,2,4-oxadiazol-3-yl)-2-methylphenyl)imidazo[1,2-a]pyridine-3-carboxamide, which show thermal events such as dehydration/desolvation and recrystallization events during DSC experiments, indicating solvent/water losses and phase transformations. It is worth mentioning that Form A and Form B of N-(5-(5-((1R,2S)-2-fluorocyclopropyl)-1,2,4-oxadiazol-3-yl)-2-methylphenyl)imidazo[1,2-a]pyridine-3-carboxamide appear to at least partially transform to N-(5-(5-((1R,2S)-2-fluorocyclopropyl)-1,2,4-oxadiazol-3-yl)-2-methylphenyl)imidazo[1,2-a]pyridine-3-carboxamide Form A during DSC experiments, which is indicated by the final melting endotherm having a peak temperature of about 175° C., which can be assigned to the melting of N-(5-(5-((1R,2S)-2-fluorocyclopropyl)-1,2,4-oxadiazol-3-yl)-2-methylphenyl)imidazo[1,2-a]pyridine-3-carboxamide Form A.

Hence, the thermal stability of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention is superior compared to the hydrated Forms HA and HB of N-(5-(5-((1R,2S)-2-fluorocyclopropyl)-1,2,4-oxadiazol-3-yl)-2-methylphenyl)imidazo[1,2-a]pyridine-3-carboxamide.

Composition

In another aspect, the present invention relates to a composition comprising a crystalline form of N-(5-(5-((1R,2S)-2-fluorocyclopropyl)-1,2,4-oxadiazol-3-yl)-2-methylphenyl)imidazo[1,2-a]pyridine-3-carboxamide, where the crystalline form can be Form A, Form HA or Form HB or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention as defined in any of the embodiments described above.

In another aspect, the present invention relates to a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention as defined in any of the embodiments described above, said composition being essentially free of any other solid-state form of N-(5-(5-((1R,2S)-2-fluorocyclopropyl)-1,2,4-oxadiazol-3-yl)-2-methylphenyl)imidazo[1,2-a]pyridine-3-carboxamide. For example, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention comprises at most 20 weight %, preferably at most 10 weight %, more preferably at most 5 weight %, 4 weight %, 3 weight %, 2 weight % or 1 weight % of any other solid-state form of N-(5-(5-((1R,2S)-2-fluorocyclopropyl)-1,2,4-oxadiazol-3-yl)-2-methylphenyl)imidazo[1,2-a]pyridine-3-carboxamide, based on the weight of the composition. The any other solid-state form of N-(5-(5-((1R,2S)-2-fluorocyclopropyl)-1,2,4-oxadiazol-3-yl)-2-methylphenyl)imidazo[1,2-a]pyridine-3-carboxamide can be Form HA of N-(5-(5-((1R,2S)-2-fluorocyclopropyl)-1,2,4-oxadiazol-3-yl)-2-methylphenyl)imidazo[1,2-a]pyridine-3-carboxamide which has a PXRD comprising amongst others characteristic reflections at 2-Theta angles of (12.8±0.2) ° and (13.6±0.2)°, when measured at a temperature in the range of from 20 to 30° C. with Cu-Kalpha radiation having a wavelength of 0.15406 nm. Therefore, the absence of reflections at 2-Theta angles of (12.8±0.2)° and (13.6±0.2)° in the PXRD confirms the absence of N-(5-(5-((1R,2S)-2-fluorocyclopropyl)-1,2,4-oxadiazol-3-yl)-2-methylphenyl)imidazo[1,2-a]pyridine-3-carboxamide form HA in the composition. In addition, the any other solid-state form of N-(5-(5-((1R,2S)-2-fluorocyclopropyl)-1,2,4-oxadiazol-3-yl)-2-methylphenyl)imidazo[1,2-a]pyridine-3-carboxamide can be Form HB of N-(5-(5-((1R,2S)-2-fluorocyclopropyl)-1,2,4-oxadiazol-3-yl)-2-methylphenyl)imidazo[1,2-a]pyridine-3-carboxamide which has a PXRD comprising amongst others characteristic reflections at 2-Theta angles of (6.7±0.2)° and (18.0±0.2)°, when measured at a temperature in the range of from 20 to 30° C. with Cu-Kalpha radiation having a wavelength of 0.15406 nm. Therefore, the absence of reflections at 2-Theta angles of (6.7±0.2) ° and (18.0±0.2)° in the PXRD confirms the absence of N-(5-(5-((1R,2S)-2-fluorocyclopropyl)-1,2,4-oxadiazol-3-yl)-2-methylphenyl)imidazo[1,2-a]pyridine-3-carboxamide form HB in the composition.

If a composition comprising N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, preferably the any other solid-state form of N-(5-(5-((1R,2S)-2-fluorocyclopropyl)-1,2,4-oxadiazol-3-yl)-2-methylphenyl)imidazo[1,2-a]pyridine-3-carboxamide is form A.

Hence, in a preferred embodiment, the present invention relates to a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention as defined in any of the embodiments described above, said composition having a PXRD comprising no reflections at 2-Theta angles of (12.8±0.2)° and (13.6±0.2)°, when measured at a temperature in the range of from 20 to 30° C. with Cu-Kalpha radiation having a wavelength of 0.15406 nm, or said composition having a PXRD comprising no reflections at 2-Theta angles of (6.7±0.2)° and (18.0±0.2)°, when measured at a temperature in the range of from 20 to 30° C. with Cu-Kalpha radiation having a wavelength of 0.15406 nm, or said composition having a PXRD comprising no reflections at 2-Theta angles of (6.7±0.2)°, (12.8±0.2)°, (13.6±0.2) ° and (18.0±0.2)°, when measured at a temperature in the range of from 20 to 30° C. with Cu-Kalpha radiation having a wavelength of 0.15406 nm.

Furthermore, in a preferred embodiment, the present invention relates to a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention as defined in any of the embodiments described above, said composition comprising at most 20 weight %, 10 weight %, 5 weight %, 2 weight % or 1 weight % of N-(5-(5-((1R,2S)-2-fluorocyclopropyl)-1,2,4-oxadiazol-3-yl)-2-methylphenyl)imidazo[1,2-a]pyridine-3-carboxamide Form HA, based on the weight of the composition, wherein Form HA is characterized by having a powder X-ray diffractogram comprising reflections at 2-Theta angles of (12.8±0.2) ° and (13.6±0.2)°, when measured at a temperature in the range of from 20 to 30° C. with Cu-Kalpha radiation having a wavelength of 0.15406 nm.

Furthermore, in a preferred embodiment, the present invention relates to a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form HB of the present invention as defined in any of the embodiments described above, said composition comprising at most 20 weight %, 10 weight %, 5 weight %, 2 weight % or 1 weight % of N-(5-(5-((1R,2S)-2-fluorocyclopropyl)-1,2,4-oxadiazol-3-yl)-2-methylphenyl)imidazo[1,2-a]pyridine-3-carboxamide Form HA, based on the weight of the composition, wherein Form HA is characterized by having a powder X-ray diffractogram comprising reflections at 2-Theta angles of (6.7±0.2)° and (18.0±0.2)°, when measured at a temperature in the range of from 20 to 30° C. with Cu-Kalpha radiation having a wavelength of 0.15406 nm.

Hence, in a preferred embodiment, the present invention relates to a composition comprising the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention as defined in any of the embodiments described above, said composition having a powder X-ray diffractogram (PXRD) comprising no reflections at 2-Theta angles of (13.2±0.2)° and (19.7±0.2)°, when measured at a temperature in the range of from 20 to 30° C. with Cu-Kalpha radiation having a wavelength of 0.15406 nm.

Furthermore, in a preferred embodiment, the present invention relates to a composition comprising the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention as defined in any of the embodiments described above, said composition comprising at most 20 weight %, 10 weight %, 5 weight %, 2 weight % or 1 weight % of N-(5-(5-((1R,2S)-2-fluorocyclopropyl)-1,2,4-oxadiazol-3-yl)-2-methylphenyl)imidazo[1,2-a]pyridine-3-carboxamide Form A, based on the weight of the composition, wherein Form A is characterized by having a powder X-ray diffractogram (PXRD) comprising reflections at 2-Theta angles of ((13.2±0.2)° and (19.7±0.2)°, when measured at a temperature in the range of from 20 to 30° C. with Cu-Kalpha radiation having a wavelength of 0.15406 nm.

In another embodiment, the invention relates to a composition comprising at least 90 w-%, including at least 90, 91, 92, 93, 94, 95, 96, 97, 98 and 99 w-%, and also including equal to about 100 w-% of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the embodiments described above, based on the total weight of the composition. The remaining material may comprise other solid form(s) of N-(5-(5-((1R,2S)-2-fluorocyclopropyl)-1,2,4-oxadiazol-3-yl)-2-methylphenyl)imidazo[1,2-a]pyridine-3-carboxamide, and/or reaction impurities and/or processing impurities arising from the preparation of the composition.

Processes

In a further aspect, the present invention relates to a process for the preparation of the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention or the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A as defined in any one of the aspects and their corresponding embodiments described above comprising:
(i) providing N-(5-(5-((1R,2S)-2-fluorocyclopropyl)-1,2,4-oxadiazol-3-yl)-2-methylphenyl)imidazo[1,2-a]pyridine-3-carboxamide in solid form;
(ii) dissolving N-(5-(5-((1R,2S)-2-fluorocyclopropyl)-1,2,4-oxadiazol-3-yl)-2-methylphenyl)imidazo[1,2-a]pyridine-3-carboxamide provided in step (i) in a solvent under mechanical stirring at elevated temperature;
(iii) cooling the solution from (ii) to room temperature under mechanical stirring;
(iv) separating at least a part of the crystals obtained in step (iii) from the mother liquor;
(v) optionally washing the isolated crystals obtained in step (iv); and
(vi) drying the crystals obtained in step (iii) or (iv).

N-(5-(5-((1R,2S)-2-fluorocyclopropyl)-1,2,4-oxadiazol-3-yl)-2-methylphenyl)imidazo[1,2-a]pyridine-3-carboxamide can for example be prepared according to the procedure provided in example F110 of WO 2013/033070 A1. N-(5-(5-((1R,2S)-2-fluorocyclopropyl)-1,2,4-oxadiazol-3-yl)-2-methylphenyl)imidazo[1,2-a]pyridine-3-carboxamide may be applied as crystalline and/or amorphous material in step (i) of the above described procedure.

N-(5-(5-((1R,2S)-2-fluorocyclopropyl)-1,2,4-oxadiazol-3-yl)-2-methylphenyl)imidazo[1,2-a]pyridine-3-carboxamide is dissolved in a solvent while stirring, with the resulting N-(5-(5-((1R,2S)-2-fluorocyclopropyl)-1,2,4-oxadiazol-3-yl)-2-methylphenyl)imidazo[1,2-a]pyridine-3-carboxamide being in a concentration in the range of from about 20 to 60 g/L, preferably the N-(5-(5-((1R,2S)-2-fluorocyclopropyl)-1,2,4-oxadiazol-3-yl)-2-methylphenyl)imidazo[1,2-a]pyridine-3-carboxamide concentration in the solution provided in (ii) is a concentration in the range of from about 30 to 60 g/L, more preferably the N-(5-(5-((1R,2S)-2-fluorocyclopropyl)-1,2,4-oxadiazol-3-yl)-2-methylphenyl)imidazo[1,2-a]pyridine-3-carboxamide concentration in the solution provided in (ii) is a concentration in the range of from about 40 to 60 g/L, and most preferably the N-(5-(5-((1R,2S)-2-fluorocyclopropyl)-1,2,4-oxadiazol-3-yl)-2-methylphenyl)imidazo[1,2-a]pyridine-3-carboxamide concentration i in the solution provided in (ii) is about 50 g/L.

The solvent used in the solution provided in (ii) is 2-propanol, acetone, methyl tert-butyl ether (MTBE), 95% ethanol or dichloromethane (DCM). Preferably the solvent used in the solution provided in (ii) is 2-propanol.

Aside from mechanical stirring, the dissolution step (ii) can involve any kind of movement of N-(5-(5-((1R,2S)-2-fluorocyclopropyl)-1,2,4-oxadiazol-3-yl)-2-methylphenyl)imidazo[1,2-a]pyridine-3-carboxamide suspended in the solvent caused by, but not limited to e.g. agitation, mixing, shaking, vibration, sonication, wet milling and the like. The dissolution step (ii) is conducted at elevated temperature for example at a temperature in the range of from about 40 to 80° C., and then the resulting solution is allowed to cool to room temperature. Preferably, N-(5-(5-((1R,2S)-2-fluorocyclopropyl)-1,2,4-oxadiazol-3-yl)-2-methylphenyl)imidazo[1,2-a]pyridine-3-carboxamide provided in (i) is dissolved in a solvent at elevated temperature for example at a temperature in the range of from about 50 to 80° C. under mechanical stirring, and then continually stirred for 3-24 hours as the solution cools to room temperature. More preferably, N-(5-(5-((1R,2S)-2-fluorocyclopropyl)-1,2,4-oxadiazol-3-yl)-2-methylphenyl)imidazo[1,2-a]pyridine-3-carboxamide provided in (i) is dissolved in a solvent at elevated temperature for example at a temperature in the range of from about 60 to 80° C. under mechanical stirring, and then continually stirred for 3-24 hours as the solution cools to room temperature. Most preferably, N-(5-(5-((1R,2S)-2-fluorocyclopropyl)-1,2,4-oxadiazol-3-yl)-2-methylphenyl)imidazo[1,2-a]pyridine-3-carboxamide provided in (i) is dissolved in a solvent at 70° C. under mechanical stirring, and then continually stirred for 3-24 hours as the solution cools to room temperature.

Steps (ii) to (iii) may be conducted for a time sufficient that at least a substantial part, preferably all of the N-(5-(5-((1R,2S)-2-fluorocyclopropyl)-1,2,4-oxadiazol-3-yl)-2-methylphenyl)imidazo[1,2-a]pyridine-3-carboxamide starting material has converted to the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention. Preferably step (ii) is performed for a period in the range of from several hours to several days. Step (ii) may for example be performed for a period in the range of from 2 hours to 7 days. More preferably step (ii) is performed for a period in the range of from 2 hours to 40 hours. Most preferably step (ii) is performed for a period in the range of from 3 hours to 30 hours. Preferably step (iii) is performed for a period in the range of from several hours to several days. Step (iii) may for example be performed for a period in the range of from 2 hours to 7 days. More preferably step (iii) is performed for a period in the range of from 2 hours to 40 hours. Most preferably step (iii) is performed for a period in the range of from 3 hours to 30 hours. The skilled person may monitor the conversion of N-(5-(5-((1R,2S)-2-fluorocyclopropyl)-1,2,4-oxadiazol-3-yl)-2-methylphenyl)imidazo[1,2-a]pyridine-3-carboxamide to the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention by withdrawing samples from the slurry and analyzing the samples by e.g. powder X-ray diffraction.

Once the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention is obtained or preferably obtained in essentially pure form, at least a part of the crystals may be optionally separated from the mother liquor. Preferably, the crystals are separated from their mother liquor by any conventional method such as filtration, centrifugation, solvent evaporation or decantation, more preferably by filtration or centrifugation and most preferably by filtration.

In a further step the isolated crystals are washed with at least one solvent selected from the group consisting of 2-propanol, acetone, methyl tert-butyl ether (MTBE), 95% ethanol or dichloromethane (DCM). Preferably, 2-propanol is used.

The obtained crystals may then optionally be dried. Drying may be performed at a temperature in the range of from about 20 to 80° C., preferably in the range of from about 20 to 70° C. and most preferably drying is performed at 60° C. Drying may be performed for a period in the range of from about 1 to 72 hours, preferably of from about 2 to 48 hours, more preferably of from about 4 to 36 hours and most preferably of from about 6 to 24 hours. Drying may be performed at ambient pressure and/or under reduced pressure. Preferably, drying is performed at a pressure of about 100 mbar or less, more preferably of about 50 mbar or less and most preferably of about 30 mbar or less, for example a vacuum of about 25 mbar is applied for drying.

In a further aspect, the present invention relates to a process for the preparation of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention or the composition comprising the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above comprising:
(a) slurrying a powder mixture of N-(5-(5-((1R,2S)-2-fluorocyclopropyl)-1,2,4-oxadiazol-3-yl)-2-methylphenyl)imidazo[1,2-a]pyridine-3-carboxamide and fumaric acid in a solvent;
(b) heating the suspension provided in (a) under stirring;
(c) cooling the suspension in (b) to room temperature under stirring;
(d) separating at least a part of the crystals obtained in (b) or (c) from the mother liquor;
(e) washing the isolated crystals obtained in (d): and
(f) optionally, drying the crystals obtained in any one of steps (d) or (e).

N-(5-(5-((1R,2S)-2-fluorocyclopropyl)-1,2,4-oxadiazol-3-yl)-2-methylphenyl)imidazo[1,2-a]pyridine-3-carboxamide can for example be prepared according to the procedure provided in example F110 of WO 2013/033070 A1. N-(5-(5-((1R,2S)-2-fluorocyclopropyl)-1,2,4-oxadiazol-3-yl)-2-methylphenyl)imidazo[1,2-a]pyridine-3-carboxamide may be applied as crystalline and/or amorphous material in step (a) of the above described procedure. Suitable crystalline forms which may be used are for example N-(5-(5-((1R,2S)-2-fluorocyclopropyl)-1,2,4-oxadiazol-3-yl)-2-methylphenyl)imidazo[1,2-a]pyridine-3-carboxamide form A.

N-(5-(5-((1R,2S)-2-fluorocyclopropyl)-1,2,4-oxadiazol-3-yl)-2-methylphenyl)imidazo[1,2-a]pyridine-3-carboxamide and fumaric acid are combined to form a powder mixture which is then stirred. The molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid in the powder mixture is in the range of from 1.0:0.4 to 1.0:1.2, preferably of from 1.0:0.4 to 1.0:1.1, more preferably of from 1.0:0.4 to 1.0:1.05, even more preferably of from 1.0:0.5 to 1.0:1.0, and most preferably the molar ratio is 1.0:0.5. The powder mixture is then slurried with the addition of a solvent resulting in N-(5-(5-((1R,2S)-2-fluorocyclopropyl)-1,2,4-oxadiazol-3-yl)-2-methylphenyl)imidazo[1,2-a]pyridine-3-carboxamide being in a concentration in the range of from about 20 to 60 g/L, preferably the N-(5-(5-((1R,2S)-2-fluorocyclopropyl)-1,2,4-oxadiazol-3-yl)-2-methylphenyl)imidazo[1,2-a]pyridine-3-carboxamide concentration in the slurry provided in (a) is a concentration in the range of from about 30 to 60 g/L, more preferably the N-(5-(5-((1R,2S)-2-fluorocyclopropyl)-1,2,4-oxadiazol-3-yl)-2-methylphenyl)imidazo[1,2-a]pyridine-3-carboxamide concentration in the slurry provided in (a) is a concentration in the range of from about 40 to 60 g/L, and most preferably the N-(5-(5-((1R,2S)-2-fluorocyclopropyl)-1,2,4-oxadiazol-3-yl)-2-methylphenyl)imidazo[1,2-a]pyridine-3-carboxamide concentration in the slurry provided in (a) is about 50 g/L. Additionally, the fumaric acid concentration in the slurry provided in (a) is in a concentration in the range of from about 2.5 to 22 g/L, preferably the fumaric acid concentration in the slurry provided in (a) is in a concentration in the range of from about 3.5 to 22 g/L, more preferably the fumaric acid concentration in the slurry provided in (a) is in a concentration in the range of from about 5 to 22 g/L, even more preferably the fumaric acid concentration in the slurry provided in (a) is in a concentration in the range of from about 5 to 10 g/L, and most preferably the fumaric acid concentration in the slurry provided in (a) is about 7.5 g/L.

The solvent used in the suspension provided in (a) is 2-propanol, acetone, methyl tert-butyl ether (MTBE), 95% ethanol or dichloromethane (DCM). Preferably the solvent used in the suspension provided in (a) is 2-propanol.

Slurrying encompasses any kind of movement of the powder mixture suspended in solvent caused by, but not limited to e.g. agitation, stirring, mixing, shaking, vibration, sonication, wet milling and the like. The powder mixture provided in (a) is initially slurried at room temperature, further slurrying is conducted at elevated temperature for example at a temperature in the range of from about 40 to 80° C., and then further slurrying is conducted as the slurry cools to room temperature. Preferably, the powder mixture provided in (a) is initially slurried by mechanical stirring at room temperature, further slurried at elevated temperature for example at a temperature in the range of from about 50 to 80° C., and then further slurrying is conducted as the slurry cools to room temperature. More preferably, the powder mixture provided in (a) is initially slurried by mechanical stirring at room temperature, further slurried at elevated temperature for example at a temperature in the range of from about 60 to 80° C., and then further slurrying is conducted as the slurry cools to room temperature. Most preferably, the powder mixture provided in (a) is initially slurried by mechanical stirring at room temperature, further slurried at 70° C., and then further slurrying is conducted as the slurry cools to room temperature.

Slurrying may be conducted for a time sufficient that at least a substantial part, preferably all of the N-(5-(5-((1R,2S)-2-fluorocyclopropyl)-1,2,4-oxadiazol-3-yl)-2-methylphenyl)imidazo[1,2-a]pyridine-3-carboxamide starting material has converted to the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention. Preferably slurrying is performed for a period in the range of from several hours to several days. Slurrying may for example be performed for a period in the range of from 2 hours to 7 days. More preferably slurrying is performed for a period in the range of from 2 hours to 40 hours. Most preferably slurrying is performed for a period in the range of from 3 hours to 30 hours. The skilled person may monitor the conversion of N-(5-(5-((1R,2S)-2-fluorocyclopropyl)-1,2,4-oxadiazol-3-yl)-2-methylphenyl)imidazo[1,2-a]pyridine-3-carboxamide to the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention by withdrawing samples from the slurry and analyzing the samples by e.g. powder X-ray diffraction.

Once the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention is obtained or preferably obtained in essentially pure form, at least a part of the crystals may be optionally separated from the mother liquor. Preferably, the crystals are separated from their mother liquor by any conventional method such as filtration, centrifugation, solvent evaporation or decantation, more preferably by filtration or centrifugation and most preferably by filtration.

In a further step the isolated crystals are washed with at least one solvent selected from the group consisting of 2-propanol, acetone, methyl tert-butyl ether (MTBE), 95% ethanol or dichloromethane (DCM). Preferably, 2-propanol is used.

The obtained crystals may then optionally be dried. Drying may be performed at a temperature in the range of from about 20 to 80° C., preferably in the range of from about 20 to 70° C. and most preferably drying is performed at 60° C. Drying may be performed for a period in the range of from about 1 to 72 hours, preferably of from about 2 to 48 hours, more preferably of from about 4 to 36 hours and most preferably of from about 6 to 24 hours.

Drying may be performed at ambient pressure and/or under reduced pressure. Preferably, drying is performed at a pressure of about 100 mbar or less, more preferably of about 50 mbar or less and most preferably

Pharmaceutical Compositions and Use

In a further aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention or the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above for the preparation of a pharmaceutical composition.

In a further aspect, the present invention relates to a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5(5((1R,2S)-2-fluorocyclopropyl)-1,2,4-oxadiazol-3-yl)-2-methylphenyl)imidazo[1,2-a]pyridine-3-carboxamide fumaric acid co-crystal of the present invention or the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above, preferably in an effective and/or predetermined amount, and at least one pharmaceutically acceptable excipient.

Preferably, the predetermined and/or effective amount of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the composition comprising of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above is in the range of from about 10 mg to about 500 mg as calculated as N-(5-(5-((1R,2S)-2-fluorocyclopropyl)-1,2,4-oxadiazol-3-yl)-2-methylphenyl)imidazo[1,2-a]pyridine-3-carboxamide. For example predetermined and/or effective amount of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the composition comprising of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above is 10 mg, 20 mg, 50 mg, 100 mg, 150 mg, 200 mg, 250 mg, 300 mg, 350 mg, 400 mg, 450 mg or 500 mg, preferably 50 mg, 100 mg, 300 mg or 500 mg and most preferably 300 or 500 mg calculated as N-(5-(5-((1R,2S)-2-fluorocyclopropyl)-1,2,4-oxadiazol-3-yl)-2-methylphenyl)imidazo[1,2-a]pyridine-3-carboxamide. Such doses may be for oral administration and may be for daily administration (e.g. once or twice daily administration).

The at least one pharmaceutically acceptable excipient, which is comprised in the pharmaceutical composition of the present invention, is preferably selected from the group consisting of fillers, diluents, binders, disintegrants, lubricants, glidants and combinations thereof.

In a preferred embodiment, the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above is an oral solid dosage form. Preferably the oral solid dosage form is selected from the group consisting of tablets, capsules, etc. In a particular preferred embodiment, the oral dosage form is a tablet or a capsule, most preferably a tablet.

The tablet may be prepared by mixing the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above with at least one excipient such as fillers, diluents, binders, disintegrants, lubricants, glidants or combinations thereof. Optionally, a granulation step such as a dry or wet granulation step is performed before compression.

The capsule may be prepared by mixing the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the aspects and their corresponding embodiments described above with at least one excipient such as fillers, diluents, binders, disintegrants, lubricants, glidants or combinations thereof and filling the blend into a capsule. The capsule shell may be a gelatin shell or a hydroxypropylmethylcellulose (HPMC) shell.

In a further aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use as a medicament.

In yet another aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the treatment and/or prophylaxis of a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) and primary pulmonary hypertension (PPH). In particular, the treatment and/or prophylaxis of asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type II diabetes.

In yet another aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocydopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the treatment and/or prophylaxis of asthma or allergic rhinitis.

In yet another aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocydopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the treatment and/or prophylaxis of pulmonary arterial hypertension (PAH).

In yet another aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the treatment and/or prophylaxis of irritable bowel syndrome (IBS) or inflammatory bowel disease (IBD).

In yet another aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the treatment and/or prophylaxis of urticaria, dermatosis, atopic dermatitis or allergic contact dermatitis.

In yet another aspect, the present invention relates to the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the treatment and/or prophylaxis of urticaria.

In yet another aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the manufacture of a medicament for the treatment and/or prophylaxis of a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) and primary pulmonary hypertension (PPH). In particular, the treatment and/or prophylaxis of asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type 11 diabetes.

In yet another aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the manufacture of a medicament for the treatment and/or prophylaxis of asthma or allergic rhinitis.

In yet another aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the manufacture of a medicament for the treatment and/or prophylaxis of pulmonary arterial hypertension (PAH).

In yet another aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the manufacture of a medicament for the treatment and/or prophylaxis of irritable bowel syndrome (IBS) or inflammatory bowel disease (IBD).

In yet another aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the manufacture of a medicament for the treatment and/or prophylaxis of urticaria, dermatosis, atopic dermatitis or allergic contact dermatitis.

In yet another aspect, the present invention relates to the use of the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for use in the manufacture of a medicament for the treatment and/or prophylaxis of urticaria.

In yet another aspect, the present invention relates to the use of N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) and primary pulmonary hypertension (PPH). In particular, the treatment and/or prophylaxis of asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type II diabetes.

In yet another aspect, the present invention relates to the use of N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of asthma or allergic rhinitis.

In yet another aspect, the present invention relates to the use of N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of pulmonary arterial hypertension (PAH).

In yet another aspect, the present invention relates to the use of N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of irritable bowel syndrome (IBS) or inflammatory bowel disease (IBD).

In yet another aspect, the present invention relates to the use of N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocydopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of urticaria, dermatosis, atopic dermatitis or allergic contact dermatitis.

In yet another aspect, the present invention relates to the use of N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of urticaria.

In yet another aspect, the present invention relates to a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) and primary pulmonary hypertension (PPH). In particular, the treatment and/or prophylaxis of asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type II diabetes.

In yet another aspect, the present invention relates to a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of asthma or allergic rhinitis.

In yet another aspect, the present invention relates to a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of pulmonary arterial hypertension (PAH).

In yet another aspect, the present invention relates to a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of irritable bowel syndrome (IBS) or inflammatory bowel disease (IBD).

In yet another aspect, the present invention relates to a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of urticaria, dermatosis, atopic dermatitis or allergic contact dermatitis.

In yet another aspect, the present invention relates to a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, the composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or the pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments for the treatment and/or prophylaxis of urticaria.

In yet another aspect, the present invention relates to a method of treating a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) and primary pulmonary hypertension (PPH), comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

In yet another aspect, the present invention relates to a method of treating asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type II diabetes, comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocydopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

In yet another aspect, the present invention relates to a method of treating asthma or allergic rhinitis, comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

In yet another aspect, the present invention relates to a method of treating pulmonary arterial hypertension (PAH), comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocydopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

In yet another aspect, the present invention relates to a method of treating irritable bowel syndrome (IBS) or inflammatory bowel disease (IBD), comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

In yet another aspect, the present invention relates to a method of treating urticaria, dermatosis, atopic dermatitis or allergic contact dermatitis, comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

In yet another aspect, the present invention relates to a method of treating urticarial, comprising administering to a subject a therapeutically effective amount of a N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal, a composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal or a pharmaceutical composition comprising the N-(5-(5-((1R,2S)-2-fluorocyclopropyl)-1,2,4-oxadiazol-3-yl)-2-methylphenyl)imidazo[1,2-a]pyridine-3-carboxamide Form A or N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal as defined in any one of the above described aspects and their corresponding embodiments.

### EXAMPLES

The following non-limiting examples are illustrative for the disclosure and are not to be construed as to be in any way limiting for the scope of the invention.

### Example 1: Preparation of the N-(5-(5-((1R,2S)-2-fluorocyclopropyl)-1,2,4-oxadiazol-3-yl)-2-methylphenyl)imidazo[1,2-a]pyridine-3-carboxamide Form a of the Present Invention

N-(5-(5-((1R,2S)-2-fluorocyclopropyl)-1,2,4-oxadiazol-3-yl)-2-methylphenyl)imidazo[1,2-a]pyridine-3-carboxamide (2.0 g, e.g. prepared according to the method disclosed in example F110 of WO 2013/033070 A1) was dissolved in 40 mL of 2-propanol at 70° C. and mechanically stirred for 3 hours, resulting in a clear solution was obtained. The solution was then cooled to room temperature within 3 hours and continually stirred overnight. Precipitates were filtered and washed with 2-propanol and dried overnight at 60° C. under vacuum to obtain 1062 mg of N-(5-(5-((1R,2S)-2-fluorocyclopropyl)-1,2,4-oxadiazol-3-yl)-2-methylphenyl)imidazo[1,2-a]pyridine-3-carboxamide Form A (yield: 53%).

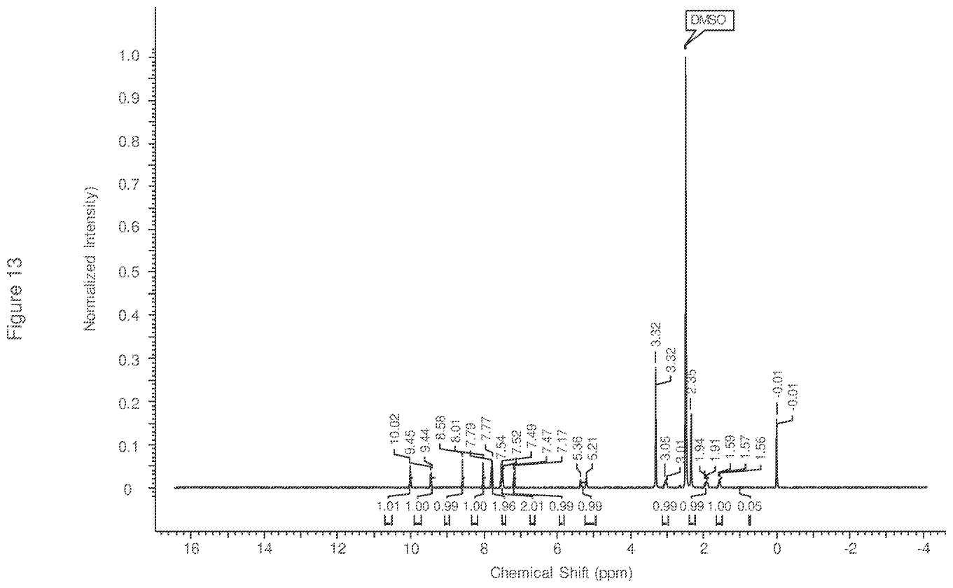

The NMR of N-(5-(5-((1R,2S)-2-fluorocyclopropyl)-1,2,4-oxadiazol-3-yl)-2-methylphenyl)imidazo[1,2-a]pyridine-3-carboxamide Form A obtained in d-DMSO using a Bruker Avance III at 400 MHz is shown in FIG. 13.

### Example 2: Powder X-Ray Diffraction-Form A

The N-(5-(5-((1R,2S)-2-fluorocyclopropyl)-1,2,4-oxadiazol-3-yl)-2-methylphenyl)imidazo[1,2-a]pyridine-3-carboxamide Form A according to the present invention was investigated by powder X-ray diffraction, which was performed with a Bruker D8 advance, Cu-Kalpha radiation (wavelength 0.15406 nm) and a Lynxeye (1D) detector.

Diffractograms were recorded at a tube voltage of 40 kV and a current of 40 mA. Step size was 0.017° with a dwell time of 0.3 s per step. Diffractograms were measured between 2-45° 2 theta.

A typical precision of the 2-Theta values is in the range of ±0.2° 2-Theta, preferably of ±0.1° 2-Theta. Thus, the diffraction peak of the N-(5-(5-((1R,2S)-2-fluorocyclopropyl)-1,2,4-oxadiazol-3-yl)-2-methylphenyl)imidazo[1,2-a]pyridine-3-carboxamide Form A of the present invention at 5.0° 2-Theta can appear in the range of from 4.8 to 5.2° 2-Theta, preferably in the range of from 4.9 to 5.1° 2-Theta on most X-ray diffractometers under standard conditions.

A representative diffractogram of the N-(5-(5-((1R,2S)-2-fluorocyclopropyl)-1,2,4-oxadiazol-3-yl)-2-methylphenyl)imidazo[1,2-a]pyridine-3-carboxamide Form A according to the present invention is displayed in FIG. 1 and the corresponding reflection list (peak list) from 3 to 30° 2-Theta and relative peak intensities are provided in table 1 below.

TABLE 1
Reflection (peak) positions of the N-(5-(5-((1R,2S)-
2-fluorocyclopropyl)-1,2,4-oxadiazol-3-yl)-2-
methylphenyl)imidazo[1,2-a]pyridine-3-carboxamide
Form A according to the present invention in the
range of from 3 to 45° 2-Theta; A typical precision of
the 2-Theta values is in the range of ± 0.2° 2-Theta,
preferably of ± 0.1° 2-Theta.

| Reflection | |
|---|---|
| position | Relative |
| [° 2-Theta] | Intensity |
| 5.0 | 11.2 |
| 8.8 | 7.6 |
| 9.8 | 29.3 |
| 10.1 | 17.5 |
| 11.4 | 3.2 |
| 13.2 | 59.7 |
| 15.2 | 100 |
| 17.1 | 17.3 |
| 17.4 | 19.4 |
| 17.6 | 14.4 |
| 18.5 | 9.3 |
| 19.7 | 68.7 |
| 20.3 | 33.0 |
| 22.1 | 39.7 |
| 22.8 | 9.2 |
| 24.5 | 12.9 |
| 25.9 | 7.9 |
| 26.7 | 4.0 |

### Example 3: Differential Scanning Calorimetry (DSC)-Form A

The N-(5-(5-((1R,2S)-2-fluorocyclopropyl)-1,2,4-oxadiazol-3-yl)-2-methylphenyl)imidazo[1,2-a]pyridine-3-carboxamide Form A according to the present invention was investigated by DSC, which was performed on a TA Discovery instrument A sample of approximately 2-4 mg was heated in an aluminum pan with pierced lid from 25 to 300° C. at a rate of 10 K/min. Nitrogen (50 mL/min) was used as purge gas.

The DSC curve of Form A (FIG. 2) shows a single endothermic peak with an onset temperature of about 175.0° C. and a peak temperature of about 175.2° C., which is due to the melting of the sample. The anhydrous and non-solvated nature of Form A and its excellent thermal stability are evidenced by the fact that neither phase changes nor desolvation events are detectable until the sample melts.

### Example 4: Thermogravimetric Analysis (TGA)-Form A

The N-(5-(5-((1R,2S)-2-fluorocyclopropyl)-1,2,4-oxadiazol-3-yl)-2-methylphenyl)imidazo[1,2-a]pyridine-3-carboxamide Form A according to the present invention was investigated by TGA, which was performed on a TA Discovery instrument. A sample of approximately 15 mg was heated in a 100 microliter aluminum pan closed with an aluminum lid. The lid was automatically pierced at the beginning of the measurement. The sample was heated from 30 to 300° C. at a rate of 10° C./min. Nitrogen (20 mL/min) was used as purge gas.

The TGA curve (FIG. 3) shows no significant mass loss until the sample melts. For example mass losses of only about 0.007 weight % up to a temperature of about 180° C. were observed, which further proves the presence of anhydrous and non-solvated Form A.

### Example 5: Preparation of the N-(5-(5-((1R,2S)-2-fluorocyclopropyl)-1,2,4-oxadiazol-3-yl)-2-methylphenyl)imidazo[1,2-a]pyridine-3-carboxamide Form HA

N-(5-(5-((1R,2S)-2-fluorocyclopropyl)-1,2,4-oxadiazol-3-yl)-2-methylphenyl)imidazo[1,2-a]pyridine-3-carboxamide Form HA was obtained from Water activity evaluation-Crystal modification analysis (Example 13-7).

### Example 6: Powder X-Ray Diffraction-Form HA

The N-(5-(5-((1R,2S)-2-fluorocyclopropyl)-1,2,4-oxadiazol-3-yl)-2-methylphenyl)imidazo[1,2-a]pyridine-3-carboxamide Form HA according to the present invention was investigated by powder X-ray diffraction, which was performed with a Bruker D8 advance, Cu-Kalpha radiation (wavelength 0.15406 nm) and a Lynxeye (1D) detector.

Diffractograms were recorded at a tube voltage of 40 kV and a current of 40 mA. Step size was 0.017° with a dwell time of 0.3 s per step. Diffractograms were measured between 2-45° 2 theta.

A typical precision of the 2-Theta values is in the range of ±0.2° 2-Theta, preferably of ±0.1° 2-Theta. Thus, the diffraction peak of the N-(5-(5-((1R,2S)-2-fluorocyclopropyl)-1,2,4-oxadiazol-3-yl)-2-methylphenyl)imidazo[1,2-a]pyridine-3-carboxamide Form HA of the present invention at 6.4° 2-Theta can appear in the range of from 6.2 to 6.6° 2-Theta, preferably in the range of from 6.3 to 6.5° 2-Theta on most X-ray diffractometers under standard conditions.

A representative diffractogram of the N-(5-(5-((1R,2S)-2-fluorocyclopropyl)-1,2,4-oxadiazol-3-yl)-2-methylphenyl)imidazo[1,2-a]pyridine-3-carboxamide Form HA according to the present invention is displayed in FIG. 4 and the corresponding reflection list (peak list) from 3 to 45° 2-Theta and relative peak intensities are provided in table 2 below.

TABLE 2
Reflection (peak) positions of the N-(5-(5-((1R,2S)-2-
fluorocyclopropyl)-1,2,4-oxadiazol-3-yl)-2-
methylphenyl)imidazo+1,2-alpyridine-3-carboxamide
Form HA according to the present invention in the
range of from 3 to 45° 2-Theta; A typical precision of
the 2-Theta values is in the range of ± 0.2° 2-Theta,
preferably of ± 0.1° 2-Theta.

| Reflection | |
|---|---|
| position | Relative |
| [° 2-Theta] | Intensity |
| 6.4 | 12.4 |
| 8.0 | 4.0 |
| 10.1 | 2.2 |
| 10.7 | 10.4 |
| 128 | 100 |
| 13.6 | 37.0 |
| 16.3 | 3.3 |
| 16.8 | 8.0 |
| 18.4 | 7.0 |
| 19.3 | 27.1 |
| 19.9 | 11.3 |
| 21.6 | 2.9 |
| 25.9 | 8.7 |
| 26.9 | 3.5 |
| 32.6 | 3.2 |

### Example 7: Differential Scanning Calorimetry (DSC)-Form HA

The N-(5-(5-((1R,2S)-2-fluorocyclopropyl)-1,2,4-oxadiazol-3-yl)-2-methylphenyl)imidazo[1,2-a]pyridine-3-carboxamide Form HA according to the present invention was investigated by DSC, which was performed on a TA Discovery instrument. A sample of approximately 2-4 mg was heated in an aluminum pan with pierced lid from 25 to 300° C. at a rate of 10 K/min. Nitrogen (50 mL/min) was used as purge gas.

The DSC curve of Form HA (FIG. 5) shows multiple thermal events, in particular melting at about 87° C., followed by melting at about 125° C., followed by recrystallization, melting at about 165° C. followed by recrystallization, and melting again with a final melting point of about 175° C. The first endotherm is dehydration followed by a melting and recrystallization (exotherm) and another melting/recrystallization event. The final melting at 175° C. is likely identical to the melting of Form A.

### Example 8: Thermogravimetric Analysis (TGA)-Form HA

The N-(5-(5-((1R,2S)-2-fluorocyclopropyl)-1,2,4-oxadiazol-3-yl)-2-methylphenyl)imidazo[1,2-a]pyridine-3-carboxamide Form HA according to the present invention was investigated by TGA, which was performed on a TA Discovery instrument. A sample of approximately 15 mg was heated in a 100 microliter aluminum pan closed with an aluminum lid. The lid was automatically pierced at the beginning of the measurement. The sample was heated from 30 to 300° C. at a rate of 10° C./min. Nitrogen (20 mL/min) was used as purge gas.

The TGA curve (FIG. 6) revealed about 5% mass loss up to a temperature of about 112° C. which further corresponds to dehydration before melting.

### Example 9: Preparation of the N-(5-(5-((1R,2S)-2-fluorocyclopropyl)-1,2,4-oxadiazol-3-yl)-2-methylphenyl)imidazo[1,2-a]pyridine-3-carboxamide Form HB

N-(5-(5-((1R,2S)-2-fluorocyclopropyl)-1,2,4-oxadiazol-3-yl)-2-methylphenyl)imidazo[1,2-a]pyridine-3-carboxamide Form HB was obtained from the Crystal modification after equilibration in water for 2 weeks study (Example 13-6).

### Example 10: Powder X-Ray Diffraction-Form HB

The N-(5-(5-((1R,2S)-2-fluorocyclopropyl)-1,2,4-oxadiazol-3-yl)-2-methylphenyl)imidazo[1,2-a]pyridine-3-carboxamide Form HB according to the present invention was investigated by powder X-ray diffraction, which was performed with a Bruker D8 advance, Cu-Kalpha radiation (wavelength 0.15406 nm) and a Lynxeye (1D) detector.

Diffractograms were recorded at a tube voltage of 40 kV and a current of 40 mA. Step size was 0.017° with a dwell time of 0.3 s per step. Diffractograms were measured between 2-45° 2 theta.

A typical precision of the 2-Theta values is in the range of ±0.2° 2-Theta, preferably of ±0.1° 2-Theta. Thus, the diffraction peak of the N-(5-(5-((1R,2S)-2-fluorocyclopropyl)-1,2,4-oxadiazol-3-yl)-2-methylphenyl)imidazo[1,2-a]pyridine-3-carboxamide Form HB of the present invention at 6.7° 2-Theta can appear in the range of from 6.5 to 6.9° 2-Theta, preferably in the range of from 6.6 to 6.8° 2-Theta on most X-ray diffractometers under standard conditions.

A representative diffractogram of the N-(5-(5-((1R,2S)-2-fluorocyclopropyl)-1,2,4-oxadiazol-3-yl)-2-methylphenyl)imidazo[1,2-a]pyridine-3-carboxamide Form HB according to the present invention is displayed in FIG. 7 and the corresponding reflection list (peak list) from 3 to 45° 2-Theta and relative peak intensities are provided in table 3 below.

TABLE 3
Reflection (peak) positions of the N-(5-(5-((1R,2S)-2-
fluorocyclopropyl)-1,2,4-oxadiazol-3-yl)-2-
methylphenyl)imidazo[1,2-a]pyrigine-3-carboxamide
Form HB according to the present invention in the range
of from 3 to 45° 2-Theta; A typical precision of the
2-Theta values is in the range of ± 0.2° 2-
Theta, preferably of ± 0.1° 2-Theta

| Reflection | |
|---|---|
| position | Relative |
| [° 2-Theta] | Intensity |
| 6.7 | 32.2 |
| 10.1 | 27.0 |
| 10.7 | 24.1 |
| 11.2 | 13.3 |
| 13.6 | 100 |
| 16.5 | 15.4 |
| 18.0 | 73.3 |
| 19.1 | 56.6 |
| 20.2 | 24.0 |
| 23.5 | 35.1 |
| 23.8 | 45.8 |
| 25.0 | 42.4 |
| 26.4 | 54.7 |
| 28.7 | 19.3 |
| 29.7 | 34.5 |

### Example 11: Differential Scanning Calorimetry (DSC)-Form HB

The N-(5-(5-((1R,2S)-2-fluorocyclopropyl)-1,2,4-oxadiazol-3-yl)-2-methylphenyl)imidazo[1,2-a]pyridine-3-carboxamide Form HB according to the present invention was investigated by DSC, which was performed on a TA Discovery instrument. A sample of approximately 2-4 mg was heated in an aluminum pan with pierced lid from 25 to 300° C. at a rate of 10 K/min. Nitrogen (50 mL/min) was used as purge gas.

The DSC curve of Form HB (FIG. 8) shows multiple thermal events, in particular melting at about 110° C., followed by recrystallisation, melting at about 125° C. followed by recrystallization, melting at about 165° C. followed by recrystallization, and melting again with a final melting point of about 173° C. The first endotherm is dehydration followed recrystallization (exotherm) and then multiple conversion. The final melting at 173° C. is likely identical to the melting of Form A.

### Example 12: Thermogravimetric Analysis (TGA)-Form HB

The N-(5-(5-((1R,2S)-2-fluorocyclopropyl)-1,2,4-oxadiazol-3-yl)-2-methylphenyl)imidazo[1,2-a]pyridine-3-carboxamide Form HB according to the present invention was investigated by TGA, which was performed on a TA Discovery instrument. A sample of approximately 15 mg was heated in a 100 microliter aluminum pan closed with an aluminum lid. The lid was automatically pierced at the beginning of the measurement. The sample was heated from 30 to 300° C. at a rate of 10′C/min. Nitrogen (20 mL/min) was used as purge gas.

The TGA curve (FIG. 9) revealed about 4.5% mass loss up to a temperature of about 100° C. which further corresponds to dehydration before melting.

### Example 13: Stability of Form A

### Example 13-1: Evaluation of Humidty at Elevated Temperature

(5-(5-((1R,2S)-2-fluorocyclopropyl)-1,2,4-oxadiazol-3-yl)-2-methylphenyl)imidazo[1,2-a]pyridine-3-carboxamide Form A (10 mg) was placed in an open vial in a 75% relative humidity (RH) chamber at 50° C. for one week and also in an open vial in a 75% relative humidity (RH) chamber at at 80° C. for one week. The initial purity of (5-(5-((1R,2S)-2-fluorocyclopropyl)-1,2,4-oxadiazol-3-yl)-2-methylphenyl)imidazo[1,2-a]pyridine-3-carboxamide Form A was 99.7%.

The samples were examined by XRPD for physical stability determination and by HPLC for chemical stability determination indicated by the presence of degradation products (DP).

The HPLC method used is PGP-27′Tl

HPLC Method

| Instrument | Waters Aquity UPLC | |
|---|---|---|
| Column | Waters Acquity UPLC BEH shield RP18 | |
| Particle size (μm) | 1.7 | |
| Dimensions (mm) | 2.1 × 50 | |
| Temperature (° C.) | 40 | |
| Flow rate (ml/min) | 0.50 | |
| Injection volume (μl) | 1 | |
| Sample solvent | Acetonitrile/water (50:50) | |
| Sample concentration (μg/ml) | 200 | |
| Detection wavelength (nm) | 240 | |
| Mobile Phase A | 0.05% TFA in Acetonitrile/water (5:95) | |
| Mobile Phase B | 0.05% TFA in Acetonitrile/water (95:5) | |
| Run time (min) | 6 | |
| Gradient | % B | Minutes |
| | 0 | Initial |
| | 25 | 4.0 |
| | 100 | 5.2 |
| | 0 | 5.21 |
| | 0 | 6.0 |

The color (CL) of the samples was evaluated by visual observation.

The results obtained are given below and show that (5-(5-((1R,2S)-2-fluorocyclopropyl)-1,2,4-oxadiazol-3-yl)-2-methylphenyl)imidazo[1,2-a]pyridine-3-carboxamide Form A is stable under the conditions tested.

| | |
|---|---|
| DP (%) | CL |

Solid state, 1 week, 50° C., 75% RH

| | | |
|---|---|---|
| Bulk (HPLC) | 0.06 | No change |

Solid state, 1 week, 80° C., 75% RH

| | | |
|---|---|---|
| Bulk (HPLC) | 0.06 | No change |

Bulk (XRPD): No change

### Example 13-2: Evaluation of Closed Container Thermal Degradation

5-(5-((1R,2S)-2-fluorocyclopropyl)-1,2,4-oxadiazol-3-yl)-2-methylphenyl)imidazo[1,2-a]pyridine-3-carboxamide Form A (10 mg) was placed in a closed vial at 50° C. and 80° C. chamber for 1 week. The initial purity of (5-(5-((1R,2S)-2-fluorocyclopropyl)-1,2,4-oxadiazol-3-yl)-2-methylphenyl)imidazo[1,2-a]pyridine-3-carboxamide Form A was 99.7%.

The samples were examined by XRPD for physical stability determination and by HPLC for chemical stability determination indicated by the presence of degradation products (DP).

The HPLC method used is described in Example 13-1. The color (CL) of the samples was evaluated by visual observation.

The results obtained are given below and show that (5-(5-((1R,2S)-2-fluorocyclopropyl)-1,2,4-oxadiazol-3-yl)-2-methylphenyl)imidazo[1,2-a]pyridine-3-carboxamide Form A is stable under the conditions tested:

| | |
|---|---|
| DP (%) | CL |

Solid state, 1 week, 50° C., tight container

| | | |
|---|---|---|
| Bulk (HPLC) | 0.06 | No change |

Solid state, 1 week, 80° C., tight container

| | | |
|---|---|---|
| Bulk (HPLC) | 0.07 | No change |

Bulk (XRPD): No change

### Example 13-3: Evaluation of Xenon Light Exposure

5-(5-((1R,2S)-2-fluorocyclopropyl)-1,2,4-oxadiazol-3-yl)-2-methylphenyl)imidazo[1,2-a]pyridine-3-carboxamide Form A (10 mg) was placed in a container and exposed to approximately 1200 kLuxh of Xenon light at 25° C. The sample was examined by XRPD for physical stability determination and by HPLC for chemical stability determination indicated by the presence of degradation products (DP). The HPLC method used is described in Example 13-1. The color (CL) of the samples was evaluated by visual observation.

The results obtained are given below and show that (5-(5-((1R,2S)-2-fluorocyclopropyl)-1,2,4-oxadiazol-3-yl)-2-methylphenyl)imidazo[1,2-a]pyridine-3-carboxamide Form A is stable under the conditions tested:

Xenon light (approx. 1200 kLuxh, 25° C.)

| | DP (%) | CL |
|---|---|---|
| Bulk (HPLC) | 0.06 | No change |

Bulk (XRPD): No change

### Example 13-4: Evaluation of the Effect of Grinding and Granulation

5-(5-((1R,2S)-2-fluorocyclopropyl)-1,2,4-oxadiazol-3-yl)-2-methylphenyl)imidazo[1,2-a]pyridine-3-carboxamide Form A (10 mg) was placed in a container and ground with added water or ethanol. Grinding and granulation with added water or ethanol showed no change in solid state, and therefore (5-(5-((1R,2S)-2-fluorocyclopropyl)-1,2,4-oxadiazol-3-yl)-2-methylphenyl)imidazo[1,2-a]pyridine-3-carboxamide Form A is stable under the conditions tested.

### Example 13-5: Hygroscopicity

The hygroscopicity of 5-(5-((1R,2S)-2-fluorocyclopropyl)-1,2,4-oxadiazol-3-yl)-2-methylphenyl)imidazo[1,2-a]pyridine-3-carboxamide Form A was evaluated by DVS at 25° C. and various RH values. The samples was examined by XRPD for physical stability determination. The results obtained are given below in Table 4 and show that (5-(5-((1R,2S)-2-fluorocyclopropyl)-1,2,4-oxadiazol-3-yl)-2-methylphenyl)imidazo[1,2-a]pyridine-3-carboxamide Form A is stable under the conditions tested.

TABLE 4

| Relative | Sorption | Desorporption. |
|---|---|---|
| humidity (% RH) | Weight | Weight |
| by DVS at 25° C. | % change | % change |
| 0 | 0.0000 | 0.0000 |
| 10 | 0.0167 | 0.0167 |
| 20 | 0.0234 | 0.0501 |
| 30 | 0.0468 | 0.0768 |
| 40 | 0.0601 | 0.0802 |
| 50 | 0.0568 | 0.0735 |
| 60 | 0.0868 | 0.0969 |
| 70 | 0.1169 | 0.1369 |
| 80 | 0.1436 | 0.1803 |
| 90 | 0.2104 | 0.2338 |

### Example 13-6: Crystal Modification after Equilibration in Water for 2 Weeks

5-(5-((1R,2S)-2-fluorocyclopropyl)-1,2,4-oxadiazol-3-yl)-2-methylphenyl)imidazo[1,2-a]pyrindine-3-carboxamide Form A (10 mg) was placed in a vial containing water for 2 weeks and the sample was then examined by XRPD for physical stability determination. It was observed that after 2 weeks in water 5-(5-((1R,2S)-2-fluorocyclopropyl)-1,2,4-oxadiazol-3-yl)-2-methylphenyl)imidazo[1,2-a]pyridine-3-carboxamide Form A converted to 5-(5-((1R,2S)-2-fluorocyclopropyl)-1,2,4-oxadiazol-3-yl)-2-methylphenyl)imidazo[1,2-a]pyridine-3-carboxamide Form HB.

### Example 13-7: Water Activity Evaluation-Crystal Modification

5-(5-((1R,2S)-2-fluorocyclopropyl)-1,2,4-oxadiazol-3-yl)-2-methylphenyl)imidazo[1,2-a]pyrindine-3-carboxamide Form A (10 mg) was placed in a vials containing various ratios of water and ethanol and allowed to equilibrate for 2 weeks. The samples were then examined by XRPD for physical stability determination. The results are given below in Table 5 which show conversion to Form HA and Form HB occurs depending on the water activity.

TABLE 5

| Water activity | Water/ethanol (v/v) | Modification |
|---|---|---|
| 0.000 | 0.000/1.000 | No change |
| 0.099 | 0.013/0.987 | No change |
| 0.205 | 0.030/0.970 | H_{A} |
| 0.298 | 0.048/0.952 | H_{A} |
| 0.398 | 0.073/0.927 | H_{A} |
| 0.502 | 0.104/0.896 | H_{A} |
| 0.605 | 0.145/0.855 | H_{A} |
| 0.700 | 0.198/0.802 | H_{A} |
| 0.802 | 0.296/0.704 | H_{A} |
| 0.900 | 0.550/0.450 | H_{B} |
| 1.000 | 1.000/0.000 | Similar to H_{A} |

### Example 14: Solubility of Form A

5-(5-((I R,2S)-2-fluorocyclopropyl)-1,2,4-oxadiazol-3-yl)-2-methylphenyl)imidazo[1,2-a]pyridine-3-carboxamide Form A (10 mg) in various media (1 mL) were mixed in a glass vial to make a slurry. Each sample was equilibrated at 25° C. for 24 hrs, and centrifuged at 13400 r.p.m. for 3 mins with 0.2 μm membrane to separate solids from liquids. The liquid was used to measure solubility by HPLC. Table 6 provides solubility data of 5-(5-((1R,2S)-2-fluorocyclopropyl)-1,2,4-oxadiazol-3-yl)-2-methylphenyl)imidazo[1,2-a]pyridine-3-carboxamide Form A at 25° C. after 24 hours equilibration and the final pH of the sample.

TABLE 6

| | Conc. | |
|---|---|---|
| Media | mg/ml | Final pH |
| pH 1.0 (0.1N HCl) | 0.44 | 1.20 |
| pH 2.0 (0.01N HCl) | 0.21 | 2.06 |
| pH 4.7, acetate buffer | LOQ | 4.78 |
| pH 6.8, phosphate buffer | LOQ | 6.77 |
| pH 10.0, borate buffer | LOQ | 10.02 |
| Water | LOQ | 7.91 |
| SGF pH 2.0 | 0.64 | 2.10 |
| FaSSIF-V2, pH 6.5 | LOQ | 6.40 |
| FeSSIF-V2, pH 5.8 | 0.015 | 5.63 |

*LOQ (Limit of Quantitaion +/− 0.5 μg/ml)

### Example 15: Preparation of the N-(5-(5-((l R,2S)-2-fluorocyclopropyl)-1,2,4-oxadiazol-3-yl)-2-methylphenyl)imidazo[1,2-a]pyridine-3-Carboxamide Fumaric Acid Co-Crystal of the Present Invention

N-(5-(5-((1R,2S)-2-fluorocyclopropyl)-1,2,4-oxadiazol-3-yl)-2-methylphenyl)imidazo[1,2-a]pyrindine-3-carboxamide (2.0 g, e.g. prepared according to the method disclosed in example F110 of WO 2013/033070 A1) and fumaric acid (310.7 mg, commercial sample from Sigma Aldrich) were mixed in a reactor and mechanically stirred while 40 mL of 2-propanol was added to the stirred powder mixture. The suspension was then heated to 70° C. or 3 hours under stirring. No clear solution was obtained. The suspension was then cooled to room temperature within 3 hours and continually stirred overnight. Precipitates were filtered and washed with 2-propanol and dried overnight at 60° C. under vacuum to obtain 2047 mg of N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal (yield: 89%).

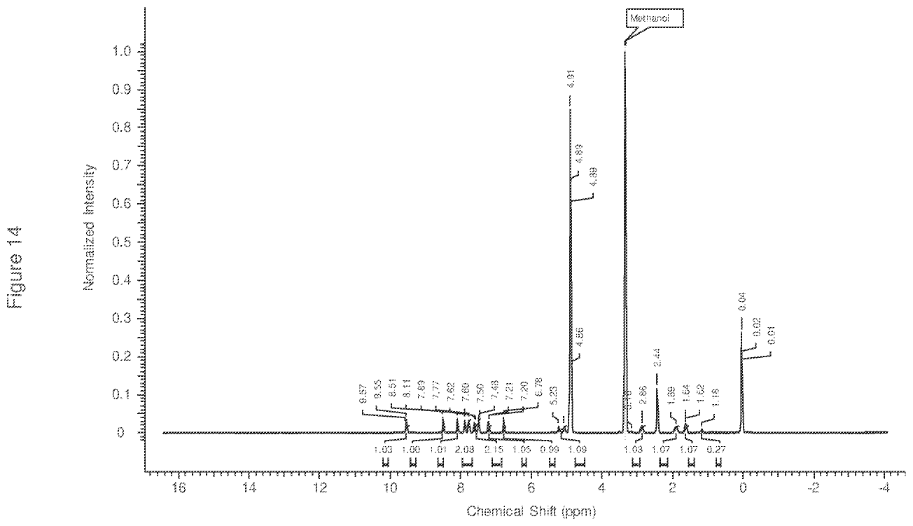

The NMR of N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal obtained using a Bruker Avance III at 400 MHz is shown in FIG. 14.

### Example 16: Powder X-Ray Diffraction

The N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention was investigated by powder X-ray diffraction, which was performed with a a Bruker D8 advance, Cu-Kalpha radiation (wavelength 0.15406 nm) and a Lynxeye (1D) detector.

Diffractograms were recorded at a tube voltage of 40 kV and a current of 40 mA. Step size was 0.017° with a dwell time of 0.3 s per step. Diffractograms were measured between 2-45° 2 theta.

A typical precision of the 2-Theta values is in the range of ±0.2° 2-Theta, preferably of ±0.1° 2-Theta. Thus, the diffraction peak of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal of the present invention at 14.9° 2-Theta can appear in the range of from 14.7 to 15.1° 2-Theta, preferably in the range of from 14.6 to 15.0° 2-Theta on most X-ray diffractometers under standard conditions.

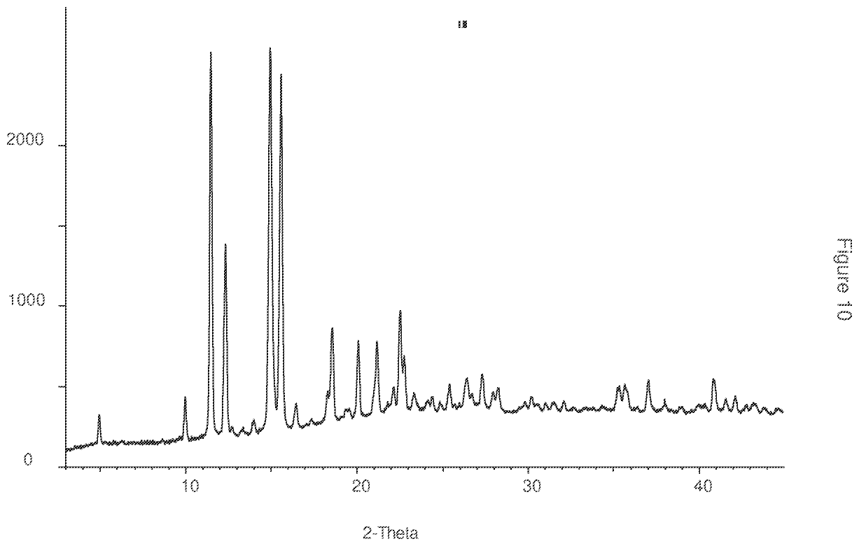

A representative diffractogram of the N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention is displayed in FIG. 10 and the corresponding reflection list (peak list) from 3 to 30° 2-Theta and relative peak intensities are provided in table 7 below.

TABLE 7
Reflection (peak) positions of the N-(5-(5-((1R,2S)-
2-fluorocyclopropyl)-1,2,4-oxadiazol-3-yl)-2-
methylphenyl)imidazo[1,2-a]pyridine-3-
carboxamide fumaric acid co-crystal according to
the present invention in the range of from 3 to
30° 2-Theta; A typical precision of the 2-Theta
values is in the range of ± 0.2° 2-Theta,
preferably of ± 0.1° 2-Theta.

| Reflection | |
|---|---|
| position | Relative |
| [° 2-Theta] | Intensity |
| 4.9 | 7.7 |
| 10.0 | 11.2 |
| 11.5 | 100. |
| 12.3 | 49.7 |
| 14.9 | 99.7 |
| 15.6 | 92.1 |
| 16.5 | 5.8 |
| 18.6 | 23.9 |
| 20.1 | 19.4 |
| 21.2 | 18.0 |
| 22.6 | 25.2 |
| 22.8 | 13.5 |
| 25.4 | 5.9 |
| 26.5 | 7.3 |

### Example 17: Differential Scanning Calorimetry (DSC)

The N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention was investigated by DSC, which was performed on a TA Discovery instrument. A sample of approximately 2-4 mg was heated in an aluminum pan with pierced lid from 25 to 300° C. at a rate of 10 K/min. Nitrogen (purge rate 50 mL/min) was used as purge gas.

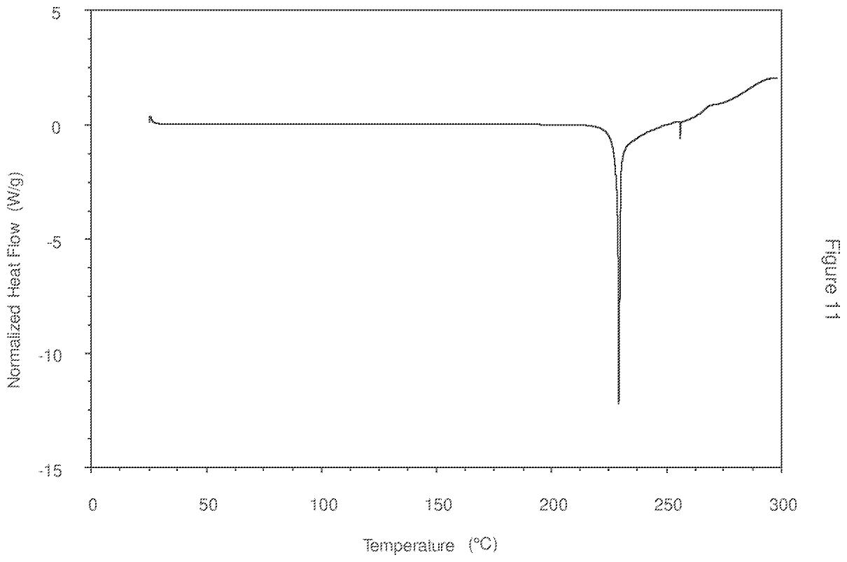

The differential scanning calorimetry curve (FIG. 11) shows a single endothermic peak with an onset temperature of about 227° C. and a peak temperature of about 229° C., which is due to the melting of the sample. The anhydrous and non-solvated nature of the co-crystal and its excellent thermal stability are evidenced by the fact that neither phase changes nor desolvation events are detectable until the sample melts.

### Example 18: Thermogravimetric Analysis (TGA)

The N-(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal according to the present invention was investigated by TGA, which was performed on a TA Discovery instrument. A sample of approximately 15 mg was heated in a 100 microliter aluminum pan closed with an aluminum lid. The lid was automatically pierced at the beginning of the measurement. The sample was heated from 30 to 300° C. at a rate of 10° C./min. Nitrogen (purge rate 20 mL/min) was used as purge gas.

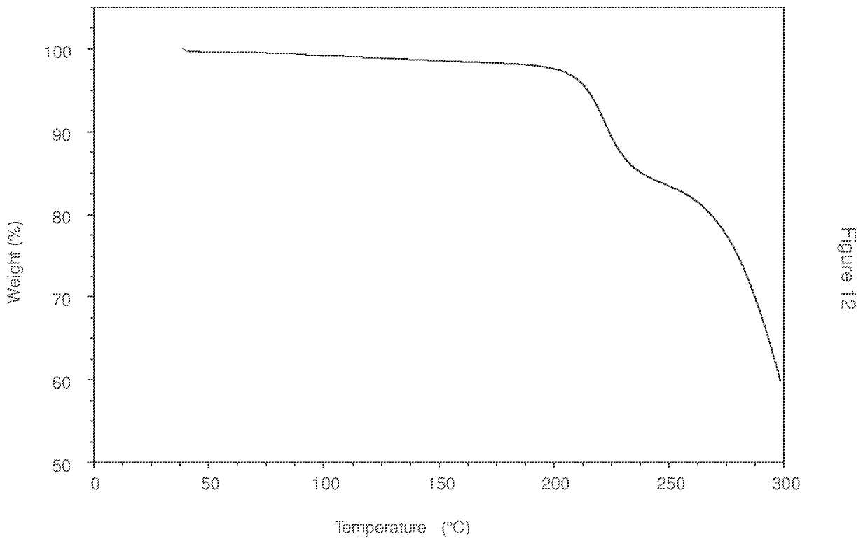

The TGA curve (FIG. 12) shows no significant mass loss until the sample melts. For example mass losses of only about 2 weight % up to a temperature of about 200° C. were observed, which further proves the presence of an anhydrous and non-solvated co-crystal.

### Example 19: Stability of (5-(5-((1R,2S)-2-fluorocyclopropyl)-1,2,4-oxadiazol-3-yl)-2-methylphenyl)imidazo[1,2-a]pyridine-3-Carboxamide Fumaric Acid Co-Crystal

### Example 19-1: Evaluation of Humidty at Elevated Temperature

(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal (10 mg) was placed in an open vial in a 75% relative humidity (RH) chamber at 50° C. for one week and also in an open vial in a 75% relative humidity (RH) chamber at 80° C. for one week. The initial purity of (5-(5-((1R,2S)-2-fluorocyclopropyl)-1,2,4-oxadiazol-3-yl)-2-methylphenyl)imidazo[1,2-a]pyridine-3-carboxamide fumaric acid co-crystal was 99.7%.

The samples were examined by XRPD for physical stability determination and by HPLC for chemical stability determination indicated by the presence of degradation products (DP). The HPLC method used is

HPLC Method

| Instrument | Waters Aquity UPLC | |
|---|---|---|
| Column | Waters Acquity UPLC BEH shield RP18 | |
| Particle size (μm) | 1.7 | |
| Dimensions (mm) | 2.1 × 50 | |
| Temperature (° C.) | 40 | |
| Flow rate (ml/min) | 0.50 | |
| Injection volume (μl) | 1 | |
| Sample solvent | Acetonitrile/water (50:50) | |
| Sample concentration (μg/ml) | 200 | |
| Detection wavelength (nm) | 240 | |
| Mobile Phase A | 0.05% TFA in Acetonitrile/water (5:95) | |
| Mobile Phase B | 0.05% TFA in Acetonitrile/water (95:5) | |
| Run time (min) | 6 | |
| Gradient | % B | Minutes |
| | 0 | Initial |
| | 25 | 4.0 |
| | 100 | 5.2 |
| | 0 | 5.21 |
| | 0 | 6.0 |

The color (CL) of the samples was evaluated by visual observation.

The results obtained are given below and show that (5-(5-((1R,2S)-2-fluorocyclopropyl)-1,2,4-oxadiazol-3-yl)-2-methylphenyl)imidazo[1,2-a]pyridine-3-carboxamide fumaric acid co-crystal is stable under the conditions tested.

| | |
|---|---|
| DP (%) | CL |

Solid state, 1 week, 50° C., 75% RH

| | | |
|---|---|---|
| Bulk (HPLC) | 0.11 | No change |

Solid state, 1 week, 80° C., 75% RH

| | | |
|---|---|---|
| Bulk (HPLC) | 0.09 | No change |

Bulk (XRPD): No change

### Example 19-2: Evaluation of Closed Container Thermal Degradation

(5-(5-((1R,2S)-2-fluorocyclopropyl)-1,2,4-oxadiazol-3-yl)-2-methylphenyl)imidazo[1,2-a]pyridine-3-carboxamide fumaric acid co-crystal (10 mg) was placed in a closed vial at 50° C. and 80° C. chamber for 1 week. The initial purity of (5-(5-((1R,2S)-2-fluorocyclopropyl)-1,2,4-oxadiazol-3-yl)-2-methylphenyl)imidazo[1,2-a]pyridine-3-carboxamide Fumaric acid co-crystal was 99.7%.

The samples were examined by XRPD for physical stability determination and by HPLC for chemical stability determination indicated by the presence of degradation products (DP).

The HPLC method used is described in Example 19-1. The color (CL) of the samples was evaluated by visual observation.

The results obtained are given below and show that (5-(5-((1R,2S)-2-fluorocyclopropyl)-1,2,4-oxadiazol-3-yl)-2-methylphenyl)imidazo[1,2-a]pyridine-3-carboxamide fumaric acid co-crystal is stable under the conditions tested:

| | |
|---|---|
| DP (%) | CL |

Solid state, 1 week, 50° C., tight container

| | | |
|---|---|---|
| Bulk (HPLC) | 0.15 | No change |

Solid state, 1 week, 80° C., tight container

| | | |
|---|---|---|
| Bulk (HPLC) | 0.08 | No change |

Bulk (XRPD): No change

### Example 19-3: Evaluation of Xenon Light Exposure

5-(5-((1R,2S)-2-fluorocyclopropyl)-1,2,4-oxadiazol-3-yl)-2-methylphenyl)imidazo[1,2-a]pyridine-3-carboxamide Fumaric acid co-crystal (10 mg) was placed in a container and exposed to approximately 1200 kLuxh of Xenon light at 25° C. The sample was examined by XRPD for physical stability determination and by HPLC for chemical stability determination indicated by the presence of degradation products (DP). The HPLC method used is described in Example 19-1. The color (CL) of the samples was evaluated by visual observation.

The results obtained are given below and show that (5-(5-((1R,2S)-2-fluorocyclopropyl)-1,2,4-oxadiazol-3-yl)-2-methylphenyl)imidazo[1,2-a]pyridine-3-carboxamide Fumaric acid co-crystal is stable under the conditions tested:

Xenon light (approx. 1200 kLuxh, 25° C.)

| | DP (%) | CL |
|---|---|---|
| Bulk (HPLC) | 0.10 | No change |

Bulk (XRPD): No change

### Example 19-4: Evaluation of the Effects of Grinding and Granulation

5-(5-((1R,2S)-2-fluorocyclopropyl)-1,2,4-oxadiazol-3-yl)-2-methylphenyl)imidazo[1,2-a]pyridine-3-carboxamide fumaric acid co-crystal (10 mg) was placed in a container and ground with added water or ethanol. Grinding and granulation with added water or ethanol showed no change in solid state, and therefore (5-(5-((1R,2S)-2-fluorocydopropyl)-1,2,4-oxadiazol-3-yl)-2-methylphenyl)imidazo[1,2-a]pyridine-3-carboxamide fumaric acid co-crystal is stable under the conditions tested.

### Example 19-5: Hygroscopicity

The hygroscopicity of 5-(5-((1R,2S)-2-fluorocyclopropyl)-1,2,4-oxadiazol-3-yl)-2-methylphenyl)imidazo[1,2-a]pyridine-3-carboxamide fumaric acid co-crystal was evaluated by DVS at 25° C. and various RH values. The samples was examined by XRPD for physical stability determination. The results obtained are given below in Table 8 and show that (5-(5-((1R,2S)-2-fluorocyclopropyl)-1,2,4-oxadiazol-3-yl)-2-methylphenyl)imidazo[1,2-a]pyridine-3-carboxamide Fumaric acid co-crystal is stable under the conditions tested.

TABLE 8

| Relative | Sorption | Desorporption. |
|---|---|---|
| humidity (% RH) | Weight | Weight |
| by DVS at 25° C. | % change | % change |
| 0 | 0.0000 | 0.0000 |
| 10 | −0.0019 | 0.0153 |
| 20 | 0.0038 | 0.0307 |
| 30 | 0.0383 | 0.0939 |
| 40 | 0.0632 | 0.0996 |
| 50 | 0.0843 | 0.1130 |
| 60 | 0.0958 | 0 1399 |
| 70 | 0.1322 | 0.1590 |
| 80 | 0.1590 | 0.1897 |
| 90 | 0.2107 | 0.1935 |

### Example 19-6: Crystal Modification after Equilibration in Water for 2 Weeks

5-(5-((1R,2S)-2-fluorocyclopropyl)-1,2,4-oxadiazol-3-yl)-2-methylphenyl)imidazo[1,2-a]pyrindine-3-carboxamide Fumaric acid co-crystal (10 mg) was placed in a vial containing water for 2 weeks and the sample was then examined by XRPD for physical stability determination. No change in XRPD was observed after 2 weeks in water.

### Example 19-7: Water Activity Evaluation-Crystal Modification

5-(5-((1R,2S)-2-fiuorocyclopropyi)-1,2,4-oxadiazol-3-yl)-2-methylphenyl)imidazo[1,2-a]pyridine-3-carboxamide fumaric acid co-crystal (10 mg) was placed in a vials containing various ratios of water and ethanol and allowed to equilibrate for 2 weeks. The samples were then examined by XRPD for physical stability determination. The results are given below in Table 9 which show no change in XRPD.

TABLE 9

| Water activity | Water/ethanol (v/v) | Modification |
|---|---|---|
| 0.000 | 0.000/1.000 | No change |
| 0.099 | 0.013/0.987 | No change |
| 0.205 | 0.030/0.970 | No change |
| 0.298 | 0.048/0.952 | No change |
| 0.398 | 0.073/0.927 | No change |
| 0.502 | 0.104/0.896 | No change |
| 0.605 | 0.145/0.855 | No change |
| 0.700 | 0.198/0.802 | No change |
| 0.802 | 0.296/0.704 | No change |
| 0.900 | 0.550/0.450 | No change |
| 1.000 | 1.000/0.000 | No change |

### Example 20: Solubility of 5-(5-((1R,2S)-2-fluorocyclopropyl)-1,2,4-oxadiazol-3-yl)-2-methylphenyl)imidazo[1,2-a]pyridine-3-carboxamide Fumaric Acid Co-Crystal

5-(5-((1R,2S)-2-fluorocyclopropyl)-1,2,4-oxadiazol-3-yl)-2-methylphenyl)imidazo[1,2-a]pyridine-3-carboxamide fumaric acid co-crystal (10 mg) in various media (1 mL) were mixed in a glass vial to make a slurry. Each sample was equilibrated at 25° C. for 24 hrs, and centrifuged at 13400 r.p.m. for 3 mins with 0.2 μm membrane to separate solids from liquids. The liquid was used to measure solubility by HPLC. Table 10 provides solubility data of 5-(5-((1R,2S)-2-fluorocyclopropyl)-1,2,4-oxadiazol-3-yl)-2-methylphenyl)imidazo[1,2-a]pyridine-3-carboxamide fumaric acid co-crystal at 25° C. after 24 hours equilibration and the final pH of the sample.

TABLE 10

| | Conc. | |
|---|---|---|
| Media | mg/ml | Final pH |
| pH 1.0 (0.1N HCl) | 0.41 | 0.95 |
| pH 2.0 (0.01N HCl) | 0.19 | 1.98 |
| pH 4.7, acetate buffer | LOQ | 4.51 |
| pH 6.8, phosphate buffer | LOQ | 5.79 |
| pH 10.0, borate buffer | LOQ | 8.76 |
| Water | LOQ | 4.26 |
| SGF pH 2.0 | 0.18 | 1.94 |
| FaSSIF-V2, pH 6.5 | LOQ | 4.66 |
| FeSSIF-V2, pH 5.8 | 0.008 | 4.75 |

*LOQ (Limit of Quantitaion: +/− 0.5 μg/ml)

1. A crystalline form of N-(5-(5-((1R,2S)-2-fluorocyclopropyl)-1,2,4-oxadiazol-3-yl)-2-methylphenyl)imidazo[1,2-a]pyridine-3-carboxamide Form A characterized by having a powder X-ray diffractogram comprising reflections at 2-Theta angles of (5.0±0.2)° and (22.1±0.2)°, when measured at a temperature in the range of from 20 to 30° C. with Cu-Kalpha radiation having a wavelength of 0.15406 nm.

2. The crystalline form according to claim 1 characterized by having a powder X-ray diffractogram comprising additional reflections at 2-Theta angles of (8.8±0.2)°, (17.4±0.2)°, (17.6±0.2) ° and (24.5±0.2)°, when measured at a temperature in the range of from 20 to 30° C. with Cu-Kalpha radiation having a wavelength of 0.15406 nm.

3. The crystalline form according to claim 1 characterized by having a powder X-ray diffractogram comprising additional reflections at 2-Theta angles of (8.8±0.2)°, (15.2±0.2)°, (17.1±0.2)°, (17.4±0.2)°, (17.6±0.2)°, (22.8±0.2) ° and (24.5±0.2)°, when measured at a temperature in the range of from 20 to 30° C. with Cu-Kalpha radiation having a wavelength of 0.15406 nm.

4. The crystalline form according to any one of claims 1 to 3 characterized by having a powder X-ray diffractogram comprising additional reflections at 2-Theta angles of (9.8±0.2)°, (10.1±0.2)°, (11.4±0.2)°, (13.2±0.2)°, (18.5±0.2)°, (19.7±0.2)°, (20.3±0.2)°, (25.9±0.2) ° and (26.7±0.2)°, when measured at a temperature in the range of from 20 to 30° C. with Cu-Kalpha radiation having a wavelength of 0.15406 nm.

5. The crystalline form according to any one of claims 1 to 4 characterized by having a differential scanning calorimetry curve comprising an endothermic peak having an peak temperature of (175.2±0.5) ° C., when measured at a heating rate of 10 K/min.

6. The crystalline form according to any one of claims 1 to 5 characterized by having a thermogravimetric analysis curve showing a mass loss of not more than 0.01 weight %, based on the weight of the crystalline form, when heated from 30° C. to 180° C. at a rate of 10 K/min.

7. The crystalline form as defined in any one of the preceeding claims characterized by showing a mass change of not more than 0.2 w-% based on the weight of the crystalline form, when measured with gravimetric moisture sorption at a relative humidity in the range of from 10 to 100% and a temperature of (25±1) ° C.

8. A salt or co-crystal of N-(5-(5-((1R,2S)-2-fluorocyclopropyl)-1,2,4-oxadiazol-3-yl)-2-methylphenyl)imidazo[1,2-a]pyridine-3-carboxamide with fumaric acid.

9. The co-crystal of claim 8 wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is 1.8-2.2:1.

10. The co-crystal of claim 8 wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is 1.9-2.1:1.

11. The co-crystal of claim 8 wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is 1.95-2.05:1.

12. The co-crystal of claim 8 wherein the molar ratio of N-(5-(5-((1R,2S)-2-fluorocyclopropyl)-1,2,4-oxadiazol-3-yl)-2-methylphenyl)imidazo[1,2-a]pyridine-3-carboxamide to fumaric acid is 2:1.

13. The co-crystal of claim 8 characterized by having the chemical structure according to formula B 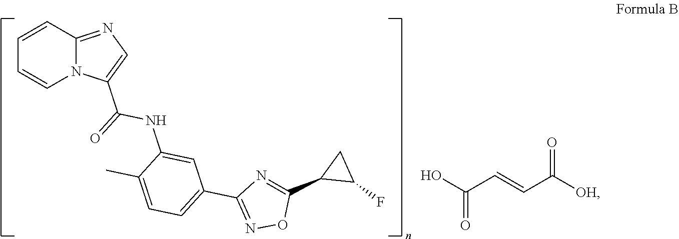
wherein n is in the range selected from 1.8 to 2.2; 1.9 to 2.1; 1.95 to 2.05 or 2.0.

14. The co-crystal according to any one claims 8-13 characterized by having a powder X-ray diffractogram (PXRD) comprising reflections at 2-Theta angles of (12.3±0.2) and (27.3±0.2)°, when measured at a temperature in the range of from 20 to 30° C. with Cu-Kalpha radiation having a wavelength of 0.15406 nm.

15. The co-crystal of claim 14 characterized by having a powder X-ray diffractogram (PXRD) comprising additional reflections at 2-Theta angles of (14.9.0±0.2)°, (16.5±0.2)°, (21.2±0.2) ° and (25.4±0.2)°, when measured at a temperature in the range of from 20 to 30° C. with Cu-Kalpha radiation having a wavelength of 0.15406 nm.

16. The co-crystal of claim 14 or claim 15 characterized by having a powder X-ray diffractogram (PXRD) comprising additional reflections at 2-Theta angles of (4.9±0.2)°, (10.0±0.2)°, (11.5±0.2)°, (15.6±0.2)°, (18.6±0.2)°, (20.1±0.2)°, (22.6±0.2)°, (22.8±0.2)° and (26.5±0.2)°, when measured at a temperature in the range of from 20 to 30° C. with Cu-Kalpha radiation having a wavelength of 0.15406 nm.

17. The co-crystal according to any one of claims 8-16 characterized by having a differential scanning calorimetry (DSC) curve comprising an endothermic peak having an onset temperature of (227±1) ° C., when measured at a heating rate of 10 K/min.

18. The co-crystal according to any one claims 8-16 characterized by having a differential scanning calorimetry (DSC) curve comprising an endothermic peak having an peak temperature of (229±1) ° C., when measured at a heating rate of 10 K/min.

19. The co-crystal according to any one of claims 8-18 characterized by having a thermogravimetric analysis (TGA) curve showing a mass loss of not more than 2.5 weight %, based on the weight of the co-crystal, when heated from 30° C. to 200° C. at a rate of 10° C./min.

20. The co-crystal as defined in any one claims 8-19 characterized by showing a mass change of not more than 0.2 w-% based on the weight of the co-crystal, when measured with gravimetric moisture sorption at a relative humidity in the range of from 10 to 100% and a temperature of (25±1) ° C.

21. A composition comprising the crystalline form as defined in any one of the preceding claims and at most 20 weight %, 10 weight %, 5 weight %, 2 weight % or 1 weight % of any other solid-state form of N-(5-(5-((1R,2S)-2-fluorocyclopropyl)-1,2,4-oxadiazol-3-yl)-2-methylphenyl)imidazo[1,2-a]pyridine-3-carboxamide, based on the weight of the composition.

22. The composition according to claim 21, wherein the crystalline form is a co-crystal of any one of claims 8-9 and the other solid-state form of N-(5-(5-((1R,2S)-2-fluorocyclopropyl)-1,2,4-oxadiazol-3-yl)-2-methylphenyl)imidazo[1,2-a]pyridine-3-carboxamide is Form A characterized by having a powder X-ray diffractogram (PXRD) comprising reflections at 2-Theta angles of (13.2±0.2)° and (19.7±0.2)°, when measured at a temperature in the range of from 20 to 30° C. with Cu-Kalpha radiation having a wavelength of 0.15406 nm.

23. The composition according to claim 21, wherein the other solid-state form of N-(5-(5-((1R,2S)-2-fluorocyclopropyl)-1,2,4-oxadiazol-3-yl)-2-methylphenyl)imidazo[1,2-a]pyridine-3-carboxamide is Form HA characterized by having a powder X-ray diffractogram comprising reflections at 2-Theta angles of (12.8±0.2)° and (13.6±0.2)°, when measured at a temperature in the range of from 20 to 30° C. with Cu-Kalpha radiation having a wavelength of 0.15406 nm.

24. The composition according to claim 21, wherein the other physical form of N-(5-(5-((1R,2S)-2-fluorocyclopropyl)-1,2,4-oxadiazol-3-yl)-2-methylphenyl)imidazo[1,2-a]pyridine-3-carboxamide is Form HB characterized by having a powder X-ray diffractogram comprising reflections at 2-Theta angles of (6.7±0.2) ° and (18.0±0.2)°, when measured at a temperature in the range of from 20 to 30° C. with Cu-Kalpha radiation having a wavelength of 0.15406 nm.

25. Use of the crystalline form as defined in any one of claims 1 to 20 or the composition as defined in any one of claims 21 to 24 for the preparation of a pharmaceutical composition.

26. A pharmaceutical composition comprising the crystalline form as defined in any one of claims 1 to 20 or the composition as defined in any one of claims 21 to 24 and at least one pharmaceutically acceptable excipient.

27. The pharmaceutical composition according to claim 26, wherein the pharmaceutical composition is an oral solid dosage form.

28. The crystalline form as defined in any one of claims 1 to 20 or the composition as defined in any one of claims 21 to 24 or the pharmaceutical composition according to any one of claims 26 to 27 for use as a medicament.

29. The crystalline form as defined in any one of claims 1 to 20 or the composition as defined in any one of claims 21 to 24 or the pharmaceutical composition according to any one of claims 26 to 27 for use in the treatment and/or prophylaxis of asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, type I diabetes or type II diabetes.

30. The crystalline form as defined in any one of claims 1 to 20 or the composition as defined in any one of claims 21 to 24 or the pharmaceutical composition according to any one of claims 26 to 27 for use in the treatment and/or prophylaxis of urticaria.

31. A process for the preparation of the crystalline form as defined in any one of claims 1 to 7 or the composition as defined in any one of claims 6 to 9 comprising:
(i) providing N-(5-(5-((1R,2S)-2-fluorocyclopropyl)-1,2,4-oxadiazol-3-yl)-2-methylphenyl)imidazo[1,2-a]pyridine-3-carboxamide in solid form;
(ii) dissolving N-(5-(5-((1R,2S)-2-fluorocyclopropyl)-1,2,4-oxadiazol-3-yl)-2-methylphenyl)imidazo[1,2-a]pyridine-3-carboxamide provided in step (i) in a solvent under mechanical stirring at elevated temperature;
(iii) cooling the solution from (ii) to room temperature under mechanical stirring;
(iv) separating at least a part of the crystals obtained in step (iii) from the mother liquor;
(v) optionally washing the isolated crystals obtained in step (iv); and
(vi) drying the crystals obtained in step (iii) or (iv).

32. A process for the preparation of the co-crystal as defined in any one of claims 8 to 20 or the composition as defined in any one of claims 18 to 21 comprising:
(a) slurrying a powder mixture of N-(5-(5-((1R,2S)-2-fluorocyclopropyl)-1,2,4-oxadiazol-3-yl)-2-methylphenyl)imidazo[1,2-a]pyridine-3-carboxamide and fumaric acid in a solvent;
(b) heating the suspension provided in (a) under stirring;
(c) cooling the suspension in (b) to room temperature under stirring;
(d) separating at least a part of the crystals obtained in (b) or (c) from the mother liquor;
(e) washing the isolated crystals obtained in (d); and
(f) optionally, drying the crystals obtained in any one of steps (d) or (e).
